(19) United States
(12) Patent Application Publication
(10) Pub. No.: US 2003/0195240 A1
(43) Pub. Date: Oct. 16, 2003

(54) Pharmaceutical compositions comprising proton pump inhibitors and gastrin/cholecystokinin receptor ligands

(76) Inventors: Sarkis Barret Kalindjian, London (GB); James Whyte Black, London (GB); Robert Anthony David Hull, London (GB); Nigel Paul Shankley, London (GB); Jean Louis Mesens, Beerse (BE); Luc Joseph Andries, Beerse (BE)

Correspondence Address: Heller Ehrman White & McAuliffe, Suite 300, 1666 K Street N W, Washington, DC, 20006, US

(21) Appl. No.: 10/275,624

(22) PCT Filed: May 4, 2001
(86) PCT No.: PCT/GB01/01963

(30) Foreign Application Priority Data
May 8, 2000 (GB) 0011098

(51) Int. Cl. A61K 31/426; A61K 31/421; A61K 31/4178

(52) U.S. Cl. 514/370; 514/397; 514/398; 514/381; 514/374

(57) ABSTRACT

Pharmaceutical compositions comprising a proton pump inhibitor and a compound of the formula (I) or its pharmaceutically acceptable salts, are useful in treating gastrointestinal disorders. X and Y are independently ═N—, —N(R⁵)— (R⁵ being selected from H, Me, Et, Pr, Bn, —OH and —CH₂COOR⁶, wherein R⁶ represents H, Me, Et, Pr or Bn), ═CH—, S— or —O—; n is from 1 to 4; R¹ is H or C₁ to C₁₅ hydrocarbyl wherein up to three C atoms may optionally be replaced by N, O and/or S atoms and up to three H atoms may optionally be replaced by halogen atoms; R² is selected from H, Me, Et, Pr and OH, each R² being independently selected from H, Me, Et, Pr and OH when n is greater than 1; R³ (when n is 1) is selected from H, Me, Et and Pr; or (when n is greater than 1) each R³ is independently selected from H, Me, Et, and Pr, or two R³ groups on neighbouring carbon atoms which are linked by a double bond; or R² and R³ on the same carbon atom are linked to form a C₃ to C₆ carbocylic ring, or two R₃ groups are absent from neighbouring carbon atoms which are linked by a double bond; or R² and R³ on the same carbon atom together represent an ═O group; R⁴ is C₁ to C₁₅ hydrocarbyl wherein up to two C atoms may optionally be replaced by N, O and/or S atoms and up to two H atoms may optionally be replaced by halogen atoms; Z is —(NR⁷)ₐ—CO—(NR⁸)_{b}— (wherein a is 0 or 1, b is 0 or 1, and R⁷ and R⁸ are independently selected from the groups recited above for R⁶), —CO—NR⁷—CH₂—CO—NR⁸—, —CO—O—, —CH₂—CH₂—, —CH═CH—, —CH₂—NR— or a bond; Q is —R⁹V, or (II) (wherein R⁹ is —CH₂—; —CH₂—CH₂—; or (III) R⁹ and R⁸, together with the nitrogen atom to which R⁸ is attached, form a piperidine or pyrrolidine ring which is substitued by V; V is —CO—NH—SO₂-Ph, SO₂—NH—CO-Ph, —CH₂OH, or a group of the formula —R¹⁰U, (wherein U is —COOH, tetrazolyl, —CONHOH— or —SO₃H; and R¹⁰ is a bond; C₁ to C₆ hydrocarbylene, optionally substituted by hydroxy, amino or acetamido; —O—(C₁ to C₃ alkylene)-; —SO₂NR¹¹—CHR¹²—; —CO—NR¹¹—CHR¹²—, R¹¹ and R¹² being independently selected from H and methyl; or —NH—(CO), —CH₂—, c being 0 or 1); T is C₁ to C₆ hydrocarbyl, —NR⁶R⁷ (wherein R⁶ and R⁷ are as defined above), —OMe, —OH, —CH₂OH, halogen or trihalomethyl; m is 1 or 2; p is from 0 to 3; and q is from 0 to 2, with the proviso that q is 1 or 2 when Z is a bond).

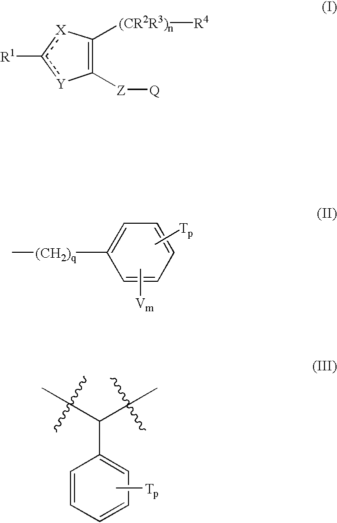

This invention relates to pharmaceutical compositions comprising a gastrin/cholecystokin (CCK) receptor ligand together with a proton pump inhibitor. (The receptor previously known as the CCK_{B}/gastrin receptor is now termed the CCK₂ receptor). This invention further relates to methods for preparing such pharmaceutical compositions.

Gastrin and the cholecystokinins are structurally related neuropeptides which exist in gastrointestinal tissue and the central nervous system (Mutt V., Gastrointestinal Hormones, Glass G. B. J., ed., Raven Press, New York, p. 169; Nisson G., ibid., p. 127).

Gastrin is one of the three primary stimulants of gastric acid secretion. Several forms of gastrin are found including 34-, 17- and 14-amino acid species with the minimum active fragment being the C-terminal tetrapeptide (TrpMetAspPhe-NH₂) which is reported in the literature to have full pharmacological activity (Tracey H. J. and Gregory R. A., Nature (London), 1964, 204, 935). Much effort has been devoted to the synthesis of analogues of this tetrapeptide (and the N-protected derivative Boc-TrpMetAspPhe-NH₂) in an attempt to elucidate the relationship between structure and activity.

Natural cholecystokinin is a 33 amino acid peptide (CCK-33), the C-terminal 5 amino acids of which are identical to those of gastrin. Also found naturally is the C-terminal octapeptide (CCK-8) of CCK-33.

The cholecystokinins are reported to be important in the regulation of appetite. They stimulate intestinal mobility, gall bladder contraction, pancreatic enzyme secretion and are known to have a trophic action on the pancreas. They also inhibit gastric emptying and have various effects in the central nervous system.

Compounds which bind to cholecystokinin and/or gastrin receptors are important because of their potential pharmaceutical use as antagonists or partial agonists of the natural peptides.

A number of gastrin antagonists have been proposed for various therapeutic applications, including the prevention of gastrin-related disorders, gastrointestinal ulcers, Zollinger-Ellison syndrome, antral G cell hyperplasia and other conditions in which lower gastrin activity or lower acid secretion is desirable. The hormone has also been shown to have a trophic action on cells and so an antagonist may be expected to be useful in the treatment of cancers, particularly in the stomach and the colon.

Possible therapeutic uses for cholecystokinin antagonists include the control of appetite disorders such as anorexia nervosa and the treatment of pancreatic inflammation, biliary tract disease and various psychiatric disorders. Other possible uses are in the potentiation of opiate (for example morphine) analgesia and in the treatment of cancers, especially of the pancreas. Moreover, ligands for cholecystokinin receptors in the brain (so-called CCK₂ receptors) have been claimed to possess anxiolytic activity.

International patent application WO93/12817, incorporated herein by reference, describes pharmaceutical compositions comprising a CCK/gastrin antagonist or a long-acting and potent H₂ antagonist and an ATP'ase proton pump inhibitor. The experimental data presented in WO93/12817 show that gastrin antagonists coadministered with proton pump inhibitors offer great therapeutic advantages.

International patent application PCT/GB99/03733, incorporated herein by reference, describes a number of gastrin and cholecystokinin (CCK) receptor ligands, methods for preparing such ligands, and compounds which are useful are useful intermediates in such methods. PCT/GB99/03733 further describes pharmaceutical compositions comprising such ligands and methods for preparing such pharmaceutical compositions. However, PCT/GB99/03733 does not describe pharmaceutical compositions comprising a gastrin/cholecystokinin (CCK) receptor ligand together with a proton pump inhibitor.

According to the present invention, there are provided pharmaceutical compositions comprising a proton pump inhibitor together with a compound of formula (I)

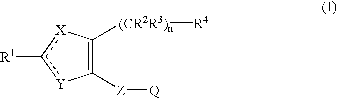

wherein X and Y are independently ═N—, —N(R⁵)— (R⁵ being selected from H, Me, Et, Pr, Bn, —OH and —CH₂COOR⁶, wherein R⁶ represents H, Me, Et, Pr or Bn), ═CH—, —S— or —O—;

n is from 1 to 4;

R¹ is H or C₁ to C₁₅ hydrocarbyl wherein up to three C atoms may optionally be replaced by N, O and/or S atoms and up to three H atoms may optionally be replaced by halogen atoms;

R² is selected from H, Me, Et, Pr and OH, each R² being independently selected from H, Me, Et, Pr and OH when n is greater than 1;

R³ (when n is 1) is selected from H, Me, Et and Pr; or (when n is greater than 1) each R³ is independently selected from H, Me, Et and Pr, or two R³ groups on neighbouring carbon atoms are linked to form a C₃ to C₆ carbocylic ring, or two R³ groups are absent from neighbouring carbon atoms which are linked by a double bond; or R² and R³ on the same carbon atom together represent an ═O group;

R⁴ is C₁ to C₁₅ is hydrocarbyl wherein up to two C atoms may optionally be replaced by N, O and/or S atoms and up to two H atoms may optionally be replaced by halogen atoms;

Z is —(NR⁷)ₐ—CO—(NR⁸)_{b}— (wherein a is 0 or 1, b is 0 or 1, and R⁷ and R⁸ are independently selected from the groups recited above for R⁶), —CO—NR⁷—CH₂—CO—NR⁸—, CO—O—, —CH₂—CH₂—, —CH═CH—, —CH₂—NR⁸— or a bond;

Q is —R⁹V, or

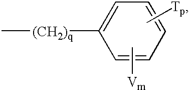

(wherein R⁹ is —CH₂—; —CH₂—CH₂—; or

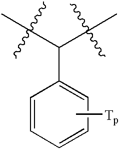

or R⁹ and R⁸, together with the nitrogen atom to which R⁸ is attached, form a piperidine or pyrrolidine ring which is substituted by V;

V is —CO—NH—SO₂-Ph, —SO₂—NH—CO-Ph, —CH₂OH, or a group of the formula —R¹⁰U, (wherein U is —COOH, tetrazolyl, —CONHOH or —SO₃H; and R¹⁰ is a bond; C₁ to C₆ hydrocarbylene, optionally substituted by hydroxy, amino or acetamido; —O—(C₁ to C₃ alkylene); —SO₂NR¹¹—CHR²—;

—CO—NR¹¹—CHR¹²—, R¹¹ and R¹² being independently selected from H and methyl; or —NH—(CO)_{c}—CH—, c being 0 or 1);

T is C₁ to C₆ hydrocarbyl, —NR⁶R⁷ (wherein R⁶ and R⁷ are as defined above), —OMe, —OH, —CH₂OH, halogen or trihalomethyl;

m is 1 or 2;

p is from 0 to 3; and

q is from 0 to 2, with the proviso that q is 1 or 2 when Z is a bond);

and pharmaceutically acceptable salts thereof;

In certain compositions according to the invention, in formula (I) R⁵ is selected from H, Me, Et, Pr and Bn; Z is (NR⁷)ₐ—CO—(NR⁸)_{b}—, —CO—NH—CH₂—CO—NH— or a bond; Q is

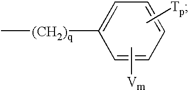

V is —CO—NH—SO₂-Ph, —SO₂—NH—CO-Ph, —OCH₂COOH, tetrazolyl or —(CH₂)ₛCOOH, wherein s is from 0 to 2; and T is C₁ to C₆ hydrocarbyl, —NR⁶R⁷, —OMe, —OH, —CH₂OH or halogen. Such compounds are disclosed in U.K. patent application No. 9824536.8, the contents of which are hereby incorporated by reference.

A further group of compositions according to the invention are those in which in formula (I) R⁵ is selected from H, Me, Et, Pr and Bn; Z is —(NR⁷)ₐ—CO—(NR⁸)_{b}—, Q is —(CH₂)ᵣCOOH, wherein r is from 1 to 3; and T is C₁ to C₆ hydrocarbyl, —NR⁶R⁷, —OMe, —OH, —CH₂OH or halogen.

A still further group of compositions according to the invention are those in which in formula (I) R⁵ is selected from H, Me, Et, Pr and Bn; -Z-Q is

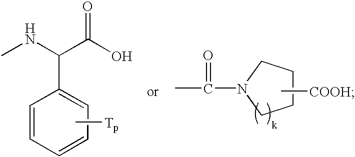

k is 1 or 2; and T is C₁ to C₆ hydrocarbyl, —NR⁶R⁷, —OMe, —OH, —CH₂OH or halogen.

Preferably X and Y are independently ═N—, ═CH—, —NH—, —NOH—, —NMe- or —NBn-. Most preferably X is —NH— or —NOH— and Y is ═CH— (or vice versa) or X is ═N— and Y is —NH— or —NOH— (or vice versa).

Preferably R¹ is C₁ to C₁₂ hydrocarbyl wherein one C atom may optionally be replaced by N or O and up to three H atoms may optionally be replaced by F, Cl or Br. More preferably R¹ is C₃ to C₁₂ alicyclic; phenyl (optionally substituted with OMe, NMe₂, CF₃, Me, F, Cl, Br or I); or C₁ to C₈ alkyl. Alicyclic groups include C₅ to C₈ cycloalkyl, C₇ to C₁₀ polycycloalkyl, C₅ to C₈ cycloalkenyl and C₇ to C₁₀ polycycloalkenyl, all optionally substituted with methyl.

Preferably Z is —CO—NH—.

Preferably Q is

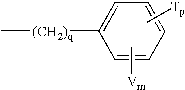

and more preferably

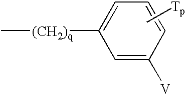

p is preferably 0 or 1, and q is preferably 0. If p is greater than 0, then T is preferably C₁ to C₆ hydrocarbyl or halo.

m is preferably 1, and V is preferably —CO₂H, —CH₂CO₂H or tetrazolyl.

Preferably R² and R³ are H; n is 1 to 3; and R⁴ is C₃ to C₁₂ carbocyclic. More preferably, R⁴ is adamantyl, cycloheptyl, cyclohexyl or phenyl. Alternatively, R⁴ may be —NH—R¹³ or —OR¹³, wherein R¹³ is C₃ to C₁₂ carbocyclic, preferably adamantyl, cycloheptyl, cyclohexyl or phenyl

R¹⁰ is preferably a bond, C₁ or C₂ alkylene (optionally substituted by hydroxy, amino or acetamido), —O—(C₁ to C₃ alkylene)-; —SO₂NR CHR¹²—; —CO—NR¹¹—CHR¹²—, —NH—(CO)_{c}—CH₂—, or a group of the formula

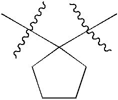

In one aspect of the present invention the proton pump inhibitor is

omeprazole which is 5-methoxy-2-[[(4-methoxy-3,5-dimethyl-2-pyridinyl)-methyl]sulfinyl]-1H-benzimidazole;

BY308;

SK & 95601 which is 2-[[(3-chloro-4-morpholino-2-pyridyl)methyl]sulfinyl]-5-methoxy-(1H)-benzimidazole;

SK & 96067 which is 3-butyryl-4-(2-methylphenylamino)-8-methoxyquinoline;

5-trifuoromethyl-2-[4-methoxy-3-methyl-2-pyridyl-methyl]-thio-[1H]-benzimidazole;

or pharmaceutically acceptable salts thereof.

These proton pump inhibitors are described and claimed in U.S. Pat. Nos. 4,472,409 and 4,255,431. These patents are incorporated herein by reference.

In a further aspect of the present invention, the proton pump inhibitor is

lansoprazole which is 2-[[[3-methyl-4-(2,2,2-trifluoroethoxy)-2-pyridinyl]methyl]sulfinyl]-1H-benzimidazole;

pantoprazole which is 5-(difluoromethoxy)-2-[[(3,4-dimethoxy-2-pyridinyl)methyl]sulfinyl]-1H-benzimidazole;

perprazole;

rabeprazole which is 2-[[4-(3-methoxypropoxy)-3-methylpyridin-2-yl]methylsulfinyl]-1H-benzimidazole;

[[4-(2,2,2-trifluoroethoxy)-3-methyl-2-pyridyl]-methyl]sulfenamide;

(Z)-5-methyl-2-[2-(1-naphthyl)ethenyl]-4-piperidinopyridine HCl;

2-(4-cyclohexyloxy-5-methylpyridin-2-yl)-3-(1-naphthyl)-1-propanol;

methyl 2-cyano-3-(ethylthio)-3-(methylthio)-2propenoate;

2-((4-methoxy-2-pyridyl)methylsulphinyl)-5-(1,1,2,2-tetrafluoroethoxy)-1H-benzimidazole sodium;

2-[[[4-(2,2,3,3,4,4,4-heptafluorobutoxy)-2-pyridyl]methyl)sulfinyl]-1H-thieno [3,4-d]imidazole;

2-[[[4-(2,2,2-trifluoroethoxy)-3-methyl-2-pyridyl]methyl]sulfinyl]-1H-benzimidazole;

2-[[[4-(2,2,2-trifluoroethoxy)-3-methyl-2-pyridyl]methyl]sulfinyl]-1H-benzimidazole;

2-methyl-8-(phenylmethoxy)-imidazo[1,2-A)-pyridine-3-acetonitrile;

(2-((2-dimethylaminobenzyl)sulfinyl)-benzimidazole);

4-(N-allyl-N-methylamino)-1-ethyl-8-((5-fluoro-6-methoxy-2-benzimidazolyl) sulfinylmethyl)-1-ethyl 1,2,3,4-tetrahydroquinolone;

2-[[(2-dimethylaminophenyl)methyl]sulfinyl]-4,7-dimethoxy-1H-benz imidazole;

2-[(2-(2-pyridyl)phenyl)sulfinyl)-1H-benzimidazole;

(2-[(2-amino-4-methylbenzyl)sulfinyl]-5-methoxybenzo[d]imidazole;

(4(2-methylpyrrol-3-yl)-2-guanidisothiazole);

4-(4-(3-(imidazole)propoxy)phenyl)-2phenylthiazole;

(E)-2-(2-(4-(3-(dipropylaniino)butoxy)phenyl)-ethenyl)benzoxazole;

(E)-2-(2-(4-(3-(dipropylamino)propoxy)phenyl)ethenyl)-benzothiazole;

Benzeneamine, 2-[[(5-methoxy-1H-benzimidazol-2-yl)sulfinyl]methyl)₄-methyl-;

Punilacidin A;

2,3-dihydro-2-methoxycarbonylamino-1,2-benzisothiazol-3-one;

2-(2-ethylaminophenylmethylsulfinyl)-5,6-dimethoxybenzimidazole;

2-methyl-8-(phenylmethoxy)imidazo[1,2-a)pyridine-3-acetonitrile;

3-amino-2-methyl-8-phenylmethoxyimidazo[1,2-a)-pyrazine HCl;

2-[[(3-chloro-4-morpholino-2-pyridyl)methyl]-sulfinyl)-5-methoxy-(1H)-benzimidazole;

[3-butyryl-4-(2-methylphenylamino)-8-methoxy-quinoline);

2-indanyl 2-(2-pyridyl)-2-thiocarbamoylacetate HCl;

2,3-dihydro-2-(2-pyridinyl)-thiazolo (3,2-a)-benzimidazole;

3-cyanomethyl-2-methyl-8-(3-methyl-2-butenyloxy)-(1,2-a)imidazopyridine;

zinc L-carnosine;

or pharmaceutically acceptable salts thereof.

Rabeprazole is described in U.S. Pat. No. 5,045,552. Lansoprazole is described in U.S. Pat. No. 4,628,098. Pantoprazole is described in U.S. Pat. No. 4,758,579. These patents are incorporated herein by reference.

Preferably, the proton pump inhibitor is selected from (RS)-rabeprazole, (RS)-omeprazole, lansoprazole, pantoprazole, (R)-omeprazole, (S)-omeprazole, perprazole, (R)-rabeprazole, (S)-rabeprazole, or the alkaline salts thereof. The alkaline salts may be, for example, the lithium, sodium, potassium, calcium or magnesium salts.

Certain compounds of the invention exist in various regioisomeric, enantiomeric, tautomeric and diastereomeric forms. It will be understood that the invention comprehends the different regioisomers, enantiomers, tautomers and diastereomers in isolation from each other as well as mixtures.

Compounds of the invention wherein

(i) X is —NH—,

(ii) Y is ═CH—, and

(iii) Z is CO—NH—

may conveniently be prepared by the route shown in Reaction Scheme A (in which PG represents a protecting group, and Q′ represents Q or a suitably protected derivative of Q):

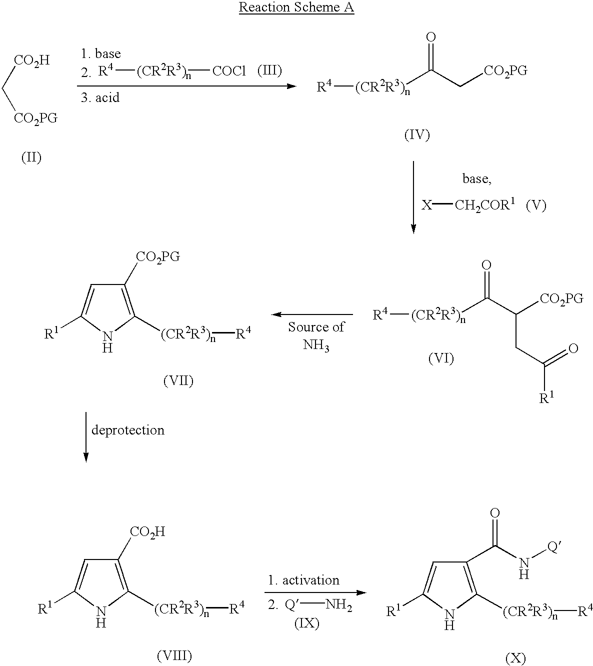

A suitably protected malonic acid derivative (II) is deprotonated and reacted with a suitably substituted acid chloride (M). The reaction product (IV) is deprotonated and reacted with a suitably substituted α-bromo carbonyl compound (V). The reaction product (VI) is cyclised, using for example AcOH and AcONH₄. The cyclisation product (VII) is deprotected to yield pyrrole (VIII). The free carboxylic acid of pyrrole (VIII) is activated, using for example SOCl₂, and reacted with a suitably substituted amine (IX) to yield compound (X). Any appropriate deprotection carried out on compounds (X) leads to compounds of the invention wherein X is —NH—, Y is ═CH— and Z is —CO—NH—.

Compounds of the invention wherein

(i) X is —NH— and Y is ═N—, or X is ═N— and Y is —NH—, and

(ii) Z is —CO—NR⁸—

may conveniently be prepared by the route shown in reaction Scheme B (in which Q′ and PG are as defined above):

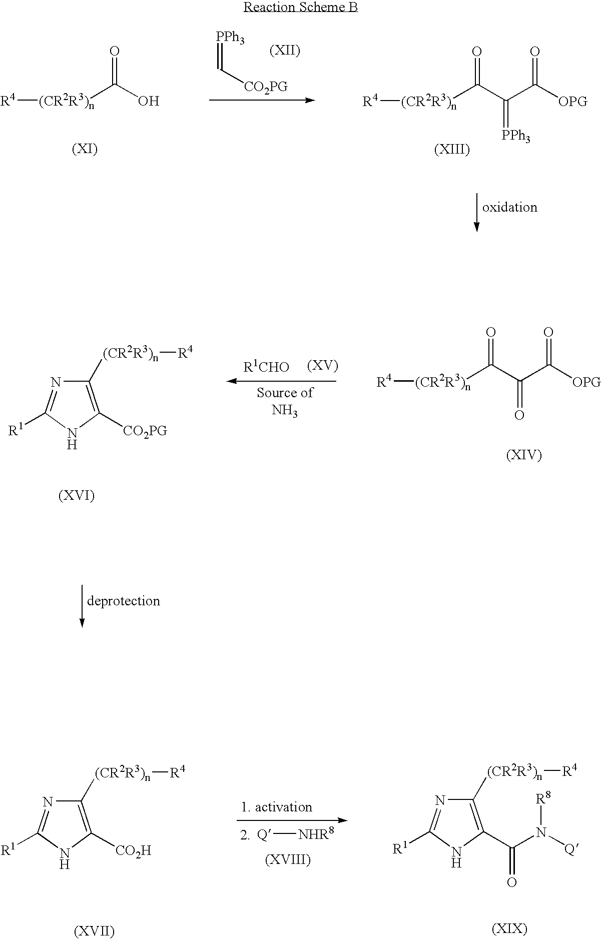

A suitably substituted carboxylic acid (XI) is reacted, using for example EDC or SOCl₂, with a suitably protected phosphorous ylid (XII). The product ylid (XIII) is oxidised, using for example oxone. The oxidation product (XIV) is cyclised with a suitably substituted aldehyde (XV), using for example AcOH and AcONH₄. The cyclisation product (XVI) is deprotected to yield imidazole (XVII). The free carboxylic acid of imidazole (XVII) is activated, using for example PyBrOP or EDC, and reacted with a suitably substituted amine (XVIII) to yield compound (XIX). Any appropriate deprotection carried out on compounds (XIX) leads to compounds of the invention wherein X is —NH— and Y is ═N— or X is ═N— and Y is —NH—, and Z is —CO—NH—.

Compounds in which X is —NR⁵— (wherein R⁵ is alkyl) may be made by treatment of compound (XVI) with sodium hydride, followed by quenching with R⁵Br, activation, reaction with Q′NHR⁸ and deprotection (if appropriate).

Hence another aspect of the present invention is a method of making a compound of formula (I). Preferably said method includes the step of cyclising a suitable precursor (VI) or (XIV) to yield a five membered ring, preferably a pyrrole (VII) or an imidazole (XVI). Preferably said cyclisation is effected using AcOH and AcONH₄. The invention further provides compounds which are useful intermediates in such methods.

Compounds of the invention wherein Z is —NH—CO—NH— or —NH—CO— may conveniently be prepared by the route shown in reaction Scheme C, in which Q′ is as defined above. X′ and Y′ correspond to X and Y, except that when X (or Y) is —NH—, X′ (or Y′, as the case may be) is —N(PG)-, in which PG represents a protecting group.

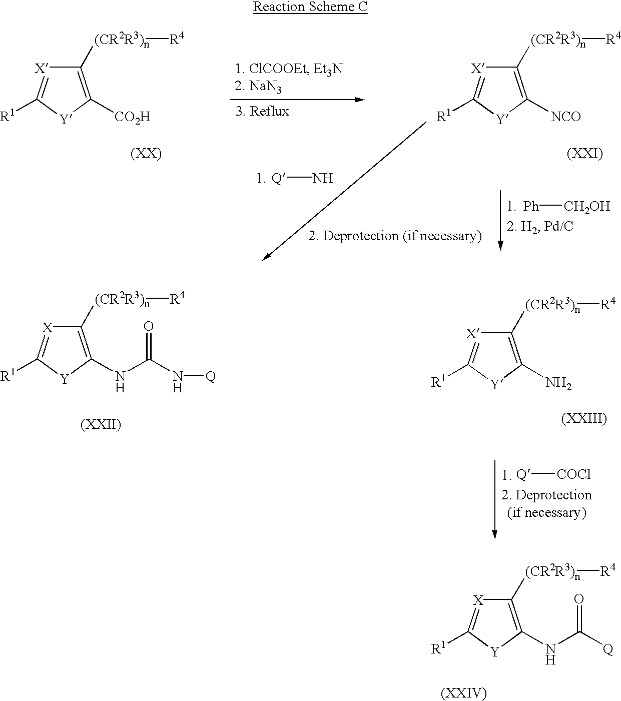

In this reaction scheme, the starting compound (XX) may be, for example, an N-protected derivative of compound (XVII) shown in Reaction Scheme B. Compound (XX) is first treated with ethylchloroformate and triethylamine, and sodium azide is then added. After heating under reflux, the compound (XXI) is obtained. Reaction of this compound with an amine of the formula Q′-NH, followed by appropriate deprotection, yields the urea derivative (XXII). Alternatively, compound (XXI) may be reacted with benzyl alcohol, followed by catalytic hydrogenation (using, for example, a Pd/C catalyst) to yield the corresponding amine (XXII). This, in turn, may be reacted with an acid chloride of the formula Q′-COCl, followed by appropriate deprotection, to provide the “reverse” amide (XXIV).

Compounds wherein Z is —CH₂—CH₂— or —CH═CH— may conveniently be prepared by the method shown in Reaction Scheme D. In this scheme, compound (XXV) is an ester derived, for example, from compound (VII) shown in Reaction Scheme A or compound (XVI) shown in Reaction Scheme B. It is first reduced to the corresponding alcohol, such as by reaction with lithium aluminium hydride, followed by oxidation (e.g. using manganese (IV) oxide) to form the corresponding aldehyde (XXVI). The aldehyde, in turn, is reacted with a triphenylphosphonium compound of the formula Q′-CH₂—PPh₃⁺Br⁻, to yield compound (XXVII). This may be deprotected as required to yield the target compound (XVI) in which Z is —CH═CH—, or it may first be reduced and then deprotected (as necessary) to provide the compound (XXIX) in which Z is

—CH₂—CH₂—.

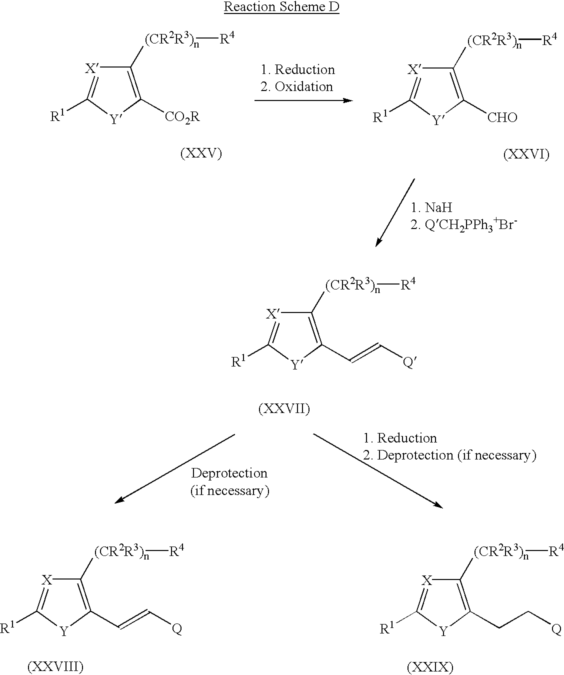

Compounds wherein X is ═N— and Y is —S— may be prepared by the procedure outlined in Reaction Scheme E.

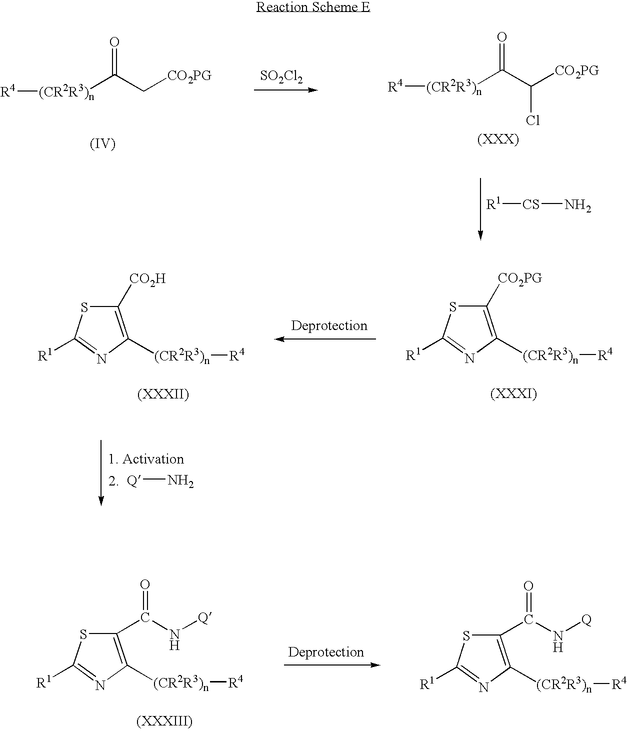

Compound (IV) (Reaction Scheme A) is first reacted with sulfonyl chloride to form compound (XX), which is then refluxed in a suitable solvent (such as ethanol) with a compound of formula R¹—CS—NH₂, leading to formation of the thiazole derivative (XXXI). This is then deprotected to form the corresponding carboxylic acid (XXXII), the carboxyl group of which may then be elaborated as shown in Reaction Schemes A, B and C. For example, amidation with a suitably protected amine Q′-NH₂ leads to compound (XXXIII), which may then be deprotected to yield the target compound. The amidation reaction is preferably carried out using PyBrOP and N,N-diisopropylethylamine.

Compounds wherein X is —S— and Y is —N— may be prepared by the method illustrated in Reaction Scheme F.

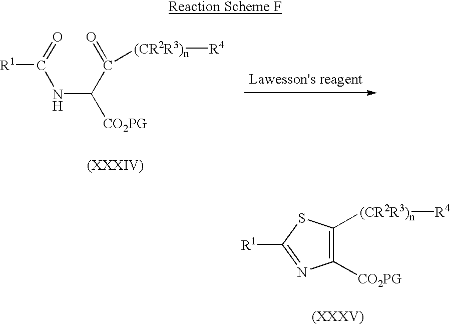

Compound (XXXIV), which may be prepared by the general methodology of Example 120, step a, is reacted with Lawesson's reagent to form the protected thiazole derivative (XXXV). This may then be deprotected, and the carboxyl group subsequently elaborated as described above.

Compound (XXXIV) may also be used in the preparation of compounds in which X is —O— and Y is ═N—, as shown in Reaction Scheme G.

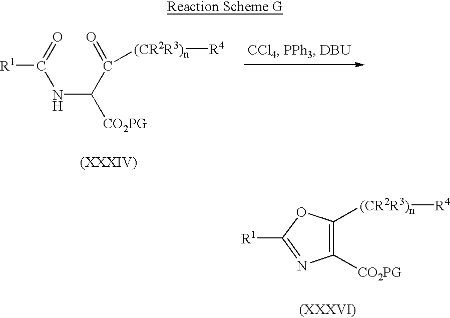

In this case, carbon tetrachloride, 1,8-diazabicyclo[5,4,0]undec-7-ene (DBU), and triphenylphosphine are sequentially added to a solution of compound (XXXIV) to form the protected oxazole derivative (XXXVI). This may then be deprotected, and the carboxyl group subsequently elaborated in the same way as for the corresponding imidazole, pyrrole and thiazole compounds.

Compounds of the invention wherein

(i) X is —N═,

(ii) Y is —N(OH)—, and

(iii) Z is CO—NR⁸—

may conveniently be prepared by the route shown in Reaction Scheme H

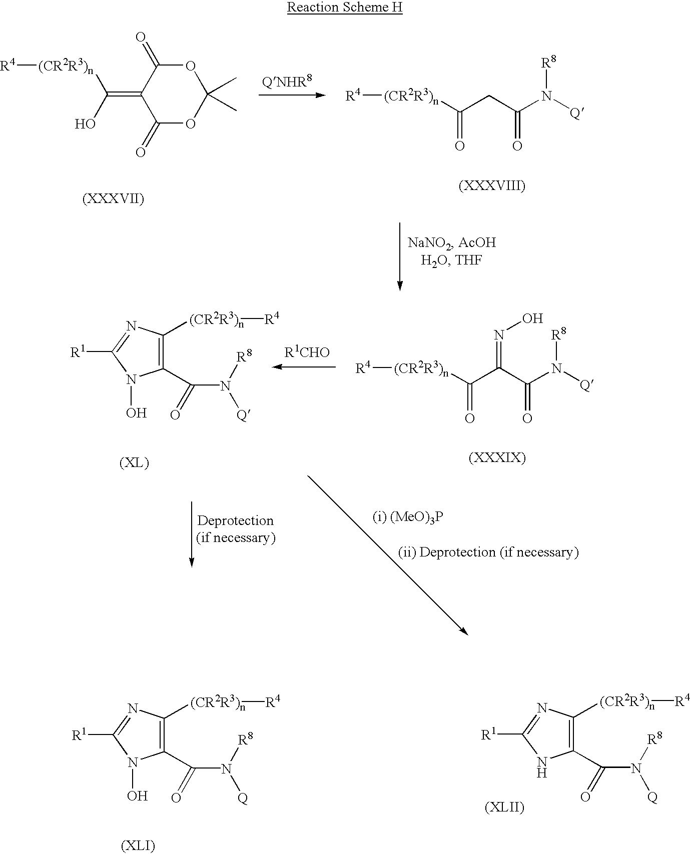

The dioxane-dione derivative (XXXVII) may be obtained by reaction of the corresponding acid R⁴—(CR²R³)ₙ—COOH with carbonyl di-imidazole, as illustrated in Example 308 below. Compound (XXXVII) is then reacted with amine Q′NHR⁸, such as by heating in toluene in the presence of catalytic quantities of DMAP. The product (XXXVI) is reacted with sodium nitrite to form the hydroxyimino derivative (XXXIX). This is then reacted with the aldehyde R¹CHO to form the substituted hydroxyimidazole (XL), which is subsequently deprotected as appropriate.

The protected hydroxyimidazole (XL) provides a further route to the corresponding imidazole compound (XLII), by treatment with trimethylphosphite, and subsequent deprotection (if necessary).

An alternative way of making intermediates which may be elaborated to compounds of the invention in which X is —N═ and Y is —NR⁵ (or vice versa) is shown in Reaction Scheme I. The protected carboxyl group of compound (XLV) may be deprotected in conventional manner, and the free carboxyl group may then be elaborated in the ways discussed above. Alternatively, compound (XLV) may be converted to compound (XVI) by reaction with trimethylphosphite.

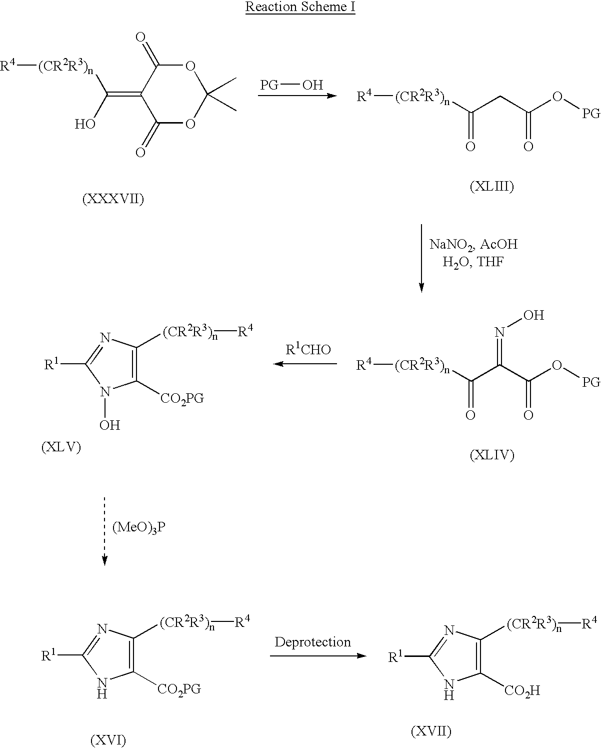

Still further routes to compound (XVI) are illustrated in Reaction Scheme J and Reaction Scheme K below.

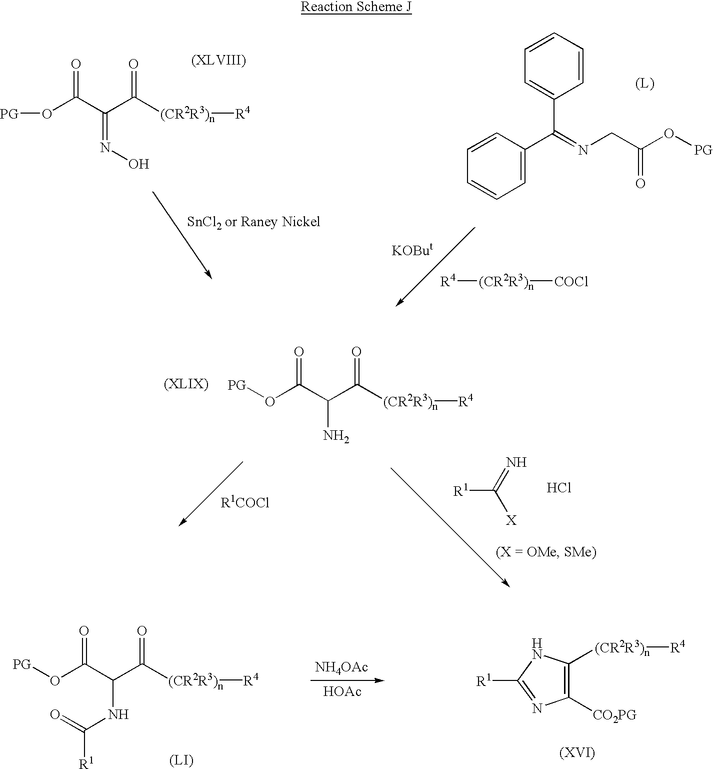

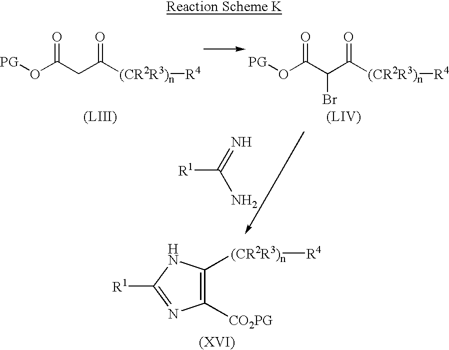

In these reaction schemes, the final product is the compound (XVI) having a protected carboxyl group. This may be deprotected and subsequently elaborated to provide compounds according to the invention, as discussed above.

The invention also comprehends derivative compounds (“pro-drugs”) which are degraded in vivo to yield the species of formula (I). Pro-drugs are usually (but not always) of lower potency at the target receptor than the species to which they are degraded. Pro-drugs are particularly useful when the desired species has chemical or physical properties which make its administration difficult or inefficient. For example, the desired species may be only poorly soluble, it may be poorly transported across the mucosal epithelium, or it may have an undesirably short plasma half-life. Further discussion of pro-drugs may be found in Stella, V. J. et al., “Prodrugs”, Drug Delivery Systems, 1985, pp. 112-176, and Drugs, 1985, 29, pp. 455473.

Pro-drug forms of the pharmacologically-active compounds of the invention will generally be compounds according to formula (I) having an acid group which is esterified or amidated. Included in such esterified acid groups are groups of the form —COOR¹⁴, wherein R¹⁴ is C₁ to C₅ alkyl, phenyl, substituted phenyl, benzyl, substituted benzyl, or one of the following:

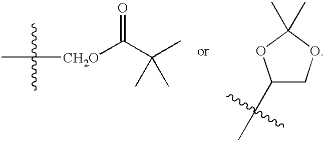

Amidated acid groups include groups of the formula —CONR¹⁵R¹⁶, wherein R¹⁵ is H, C₁ to C₅ alkyl, phenyl, substituted phenyl, benzyl, or substituted benzyl, and R¹⁶ is —OH or one of the groups just recited for R¹⁵.

Compounds of formula (I) having an amino group may be derivatised with a ketone or an aldehyde such as formaldehyde to form a Mannich base. This will hydrolyse with first order kinetics in aqueous solution.

Pharmaceutically acceptable salts of the acidic or basic compounds of the invention can of course be made by conventional procedures, such as by reacting the free base or acid with at least a stoichiometric amount of the desired salt-forming acid or base.

Pharmaceutically acceptable salts of the acidic compounds of the invention include salts with inorganic cations such as sodium, potassium, calcium, magnesium, and zinc, and salts with organic bases. Suitable organic bases include N-methyl-D-glucamine, arginine, benzathine, diolamine, olamine, procaine and tromethamine.

Pharmaceutically acceptable salts of the basic compounds of the invention include salts derived from organic or inorganic acids. Suitable anions include acetate, adipate, besylate, bromide, camsylate, chloride, citrate, edisylate, estolate, fumarate, gluceptate, gluconate, glucuronate, hippurate, hyclate, hydrobromide, hydrochloride. iodide, isethionate, lactate, lactobionate, maleate, mesylate, methylbromide, methylsulfate, napsylate, nitrate, oleate, pamoate, phosphate, polygalacturonate, stearate, succinate, sulfate, sulfosalicylate, tannate, tartrate, terephthalate, tosylate and triethiodide.

It is anticipated that the compositions of the invention can be administered by oral or parenteral routes, including intravenous, intramuscular, intraperitoneal, subcutaneous, rectal and topical administration, and inhalation.

For oral administration, the compositions of the invention will generally be provided in the form of tablets or capsules or as an aqueous solution or suspension.

Tablets for oral use may include the active ingredients mixed with pharmaceutically acceptable excipients such as inert diluents, disintegrating agents, binding agents, lubricating agents, sweetening agents, flavouring agents, colouring agents and preservatives. Suitable inert diluents include sodium and calcium carbonate, sodium and calcium phosphate and lactose. Corn starch and alginic acid are suitable disintegrating agents. Binding agents may include starch and gelatine. The lubricating agent, if present, will generally be magnesium stearate, stearic acid or talc. If desired, the tablets may be coated with a material such as glyceryl monostearate or glyceryl distearate, to delay absorption in the gastrointestinal tract.

Capsules for oral use include hard gelatine capsules in which the active ingredients are mixed with a solid diluent and soft gelatine capsules wherein the active ingredients are mixed with water or an oil such as peanut oil, liquid paraffin or olive oil.

For intramuscular, intraperitoneal, subcutaneous and intravenous use, the compositions of the invention will generally be provided in sterile aqueous solutions or suspensions, buffered to an appropriate pH and isotonicity. Suitable aqueous vehicles include Ringer's solution and isotonic sodium chloride. Aqueous suspensions according to the invention may include suspending agents such as cellulose derivatives, sodium alginate, polyvinyl-pyrrolidone and gum tragacanth, and a wetting agent such as lecithin. Suitable preservatives for aqueous suspensions include ethyl and n-propyl p-hydroxybenzoate.

Effective doses of the compositions of the present invention may be ascertained by conventional methods. The specific dosage level required for any particular patient will depend on a number of factors, including severity of the condition being treated, the route of administration and the weight of the patient. In general, however, it is anticipated that the daily dose (whether administered as a single dose or as divided doses) will be in the range 0.001 to 5000 mg per day, more usually from 1 to 1000 mg per day, and most usually from 10 to 200 mg per day. Expressed as dosage per unit body weight, a typical dose will be expected to be between 0.01 μg/kg and 50 mg/kg, especially between 10 μg/kg and 10 mg/kg, eg. between 100 μg/kg and 2 mg/kg. Preferably, the dose of each of the active ingredients will be equal to or less than that which is approved or indicated in monotherapy with said active ingredient.

In another aspect of the present invention, there is provided a kit comprising a compound of formula (I) and a proton pump inhibitor. The kit is useful as a combined preparation for simultaneous, separate or sequential use in the treatment of patients suffering from gastrointestinal disorders.

The term “hydrocarbyl” is used herein to refer to monovalent groups consisting of carbon and hydrogen. Hydrocarbyl groups thus include alkyl, alkenyl and alkynyl groups (in both straight and branched chain forms), cycloalky] (including polycycloalkyl), cycloalkenyl and aryl groups, and combinations of the foregoing, such as alkylcycloalkyl, alkylpolycycloalkyl, alkylaryl, alkenylaryl, alkynylaryl, cycloalkylaryl and cycloalkenylaryl groups.

Where reference is made to a carbon atom of a hydrocarbyl group being replaced by a N, O or S atom, what is intended is that

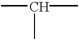

is replaced by

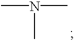

or that —CH₂— is replaced by —O— or —S—.

A “carbocyclic” group, as the term is used herein, comprises one or more closed chains or rings, which consist entirely of carbon atoms. Carbocyclic groups thus include aryl groups (such as phenyl, naphthyl, indanyl, fluorenyl, (1,2,3,4)-tetrahydronaphthyl, indenyl and isoindenyl, and substituted derivatives thereof), and also alicyclic groups. The term “alicyclic group” refers to a carbocyclic group which does not contain an aromatic ring, and thus includes groups such as cyclopropyl, cyclobutyl, cyclopentyl, cyclohexyl, cyclohexenyl, adamantyl, norbornyl, bicyclo[2.2.2]octyl, norbornenyl and bicyclo[2.2.2]octenyl, and also groups (such as adamantanemethyl and methylcyclohexyl) which contain both alkyl or alkenyl groups in addition to cycloalkyl or cycloalkenyl moieties.

The term “alkyl” is used herein to refer to both straight and branched chain forms.

The term “aryl” is used herein to refer to an aromatic group, such as phenyl or naphthyl, or a heteroaromatic group, such as pyridyl, pyrrolyl or furanyl.

A “heterocyclic” group comprises one or more closed chains or rings which have at least one atom other than carbon in the closed chain or ring. Examples include benzimidazolyl, thienyl, furanyl, pyrrolyl, imidazolyl, pyrazolyl, thiazolyl, isothiazolyl, oxazolyl, pyrrolidinyl, pyrrolinyl, imidazolidinyl, imidazolinyl, pyrazolidinyl, tetrahydrofuranyl, pyranyl, pyronyl, pyridyl, pyrazinyl, pyridazinyl, piperidyl, piperazinyl, morpholinyl, thionaphthyl, benzofuranyl, isobenzofuryl, indolyl, oxyindolyl, isoindolyl, indazolyl, indolinyl, 7-azaindolyl, isoindazolyl, benzopyranyl, coumarinyl, isocoumarinyl, quinolyl, isoquinolyl, naphthridinyl, cinnolinyl, quinazolinyl, pyridopyridyl, benzoxazinyl, quinoxadinyl, chromenyl, chromanyl, isochromanyl and carbolinyl.

When reference is made herein to a substituted carbocyclic group (such as substituted phenyl) or a substituted heterocyclic group, the substituents are preferably from 1 to 3 in number and selected from C₁ to C₆ alkyl, C₁ to C₆ alkoxy, thio, C₁ to C₆ alkylthio, carboxy, carboxy(C₁ to C₆)alkyl, formyl, C₁ to C₆ alkylcarbonyl, C₁ to C₆ alkylcarbonylalkoxy, nitro, trihalomethyl, hydroxy, C₁ to C₆ alkylhydroxy, hydroxy(C₁ to C₆)alkyl, amino, C₁ to C₆ alkylamino, di(C₁ to C₆ alkyl)amino, aminocarboxy, C₁ to C₆ alkylaminocarboxy, di(C₁ to C₆ alkyl)aminocarboxy, aminocarboxy(C₁ to C₆)alkyl, C₁ to C₆ alkylaminocarboxy(C₁ to C₆)alkyl, di(C₁ to C₆ alkyl)aminocarboxy(C₁ to C₆)alkyl, C₁ to C₆ alkylcarbonylamino, C₅ to C₈ cycloalkyl, C₅ to C₈ cycloalkyl(C₁ to C₆)alkyl, C₁ to C₆ alkylcarbonyl(C₁ to C₆ alkyl)amino, aryl, aryl(C₁ to C₆)alkyl, (C₁ to C₆ alkyl)aryl, halo, C₁ to C₆ alkylhalo, sulphamoyl, tetrazolyl and cyano.

Most usually, substituents will be selected from C₁ to C₆ alkyl, C₁ to C₆ alkoxy, thio, C₁ to C₆ alkylthio, carboxy, carboxy(C₁ to C₆)alkyl, formyl, C₁ to C₆ alkylcarbonyl, C₁ to C₆ alkylcarbonylalkoxy, nitro, trihalomethyl, hydroxy, C₁ to C₆ alkylhydroxy, hydroxy(C₁ to C₆)alkyl, amino, C₁ to C₆ alkylamino, di(C₁ to C₆ alkyl)amino, aminocarboxy, C₁ to C₆ alkylaminocarboxy, di(C₁ to C₆ alkyl)aminocarboxy, aminocarboxy(C₁ to C₆)alkyl, C₁ to C₆ alkylaminocarboxy(C₁ to C₆)alkyl, di(C₁ to C₆ alkyl)aminocarboxy(C₁ to C₆)alkyl, C₁ to C₆ alkylcarbonylamino, C₅ to C₈ cycloalkyl, C₅ to C₈ cycloalkyl(C₁ to C₆)alkyl, C₁ to C₆ alkylcarbonyl(C₁ to C₆ alkyl)amino, halo, C₁ to C₆ alkylhalo, sulphamoyl, tetrazolyl and cyano.

The term “halogen” is used herein to refer to any of fluorine, chlorine, bromine and iodine. Most usually, however, halogen substituents in the compounds of the invention are chlorine and fluorine substituents.

The invention is now further illustrated by means of the following examples of compounds according to formula (I). All reactions were performed under an atmosphere of dry argon unless otherwise stated. Anhydrous dichloromethane (DCM) was freshly distilled from calcium hydride. Anhydrous tetrahydrofuran (THF) and N,N-dimethylformamide (DMF) were used.

### EXAMPLE 1

5-[(2-Adamantan-1-ylmethyl-5-phenyl-1H-pyrrole-3-carbonyl)-amino]-isophthalic Acid

Step a. 4-Adamantan-1-yl-3-oxo-2-(2-oxo-2-phenyl-ethyl)-butyric acid ethyl ester. To a solution of 4-adamantan-1-yl-3-oxo-butyric acid ethyl ester, prepared by a modification of Wierenga and Skulnick's procedure (W. Wierenga and H. I. Skulnick, J. Org. Chem., 1979, 44, 310) (3.00 g, 11.0 mmol) in acetone (30 ml) was added sodium iodide (0.55 g, 3.67 mmol) and anhydrous potassium carbonate (3.04 g, 22.0 mmol), then a solution of 2-bromo-1-phenyl-ethanone (2.38 g, 11.5 mmol) in acetone (10 ml). The mixture was stirred at reflux for 36 h, cooled to room temperature and filtered. The filtrate was evaporated, the residue was dissolved in diethyl ether (50 ml) and washed with water (2×20 ml). The organic phase was dried (MgSO₄) and the solvent was evaporated. The residue was purified by flash column chromatography (silica, hexane/ethyl acetate 4:1) to afford the product as pale yellow oil (1.92 g, 46%). ¹H NMR (300 MHz, CDCl₃) 7.98 (2H, m), 7.65 (1H, m), 7.46 (2H, m), 4.19 (3H, m), 3.60 (1H, dd), 5.59 (1H, dd), 2.50 (2H, dd), 1.96 (3H, br s), 1.68 (12H, m), 1.29 (3H, t).

Step b. 2-Adamantan-1-ylmethyl-5-phenyl-1H-pyrrole-3-carboxylic acid ethyl ester. This compound was prepared by modification of Sammes's procedure (P.-K. Chiu and M. P. Sammes, Tetrahedron, 1990, 46, 3439). The product of step a (1.10 g, 2.88 mmol) and ammonium acetate (780 mg, 10.1 mmol) were stirred in acetic acid (1.4 ml) at 80° C. for 24 h. The reaction mixture was cooled, then partitioned between DCM and saturated sodium hydrogen carbonate. The organic layer was dried, the solvent was evaporated. The residue was crystallised from hexane/ethyl acetate 4:1 to afford the product as a white solid (750 mg, 71%). ¹H NMR (300 MHz, CDCl₃) 8.21 (1H, br s), 7.21-7.48 (5H, m), 6.88 (1H, d), 4.30 (2H, q), 2.85 (2H, s), 1.96 (3H, br s), 1.60 (12H, m), 1.38 (3H, t).

Step c. 2-Adamantan-1-ylmethyl-5-phenyl-1H-pyrrole-3-carboxylic acid. To a solution of the product of step b (750 mg, 2.06 mmol) in ethanol (45 ml) was added sodium hydroxide (5 ml of 6.0M solution). The mixture was heated at reflux for 48 h, it was allowed to cool to room temperature and concentrated to small volume under reduced pressure. The concentrated solution was diluted with 2M hydrochloric acid (40 ml), the precipitated solid was filtered, washed with water and dried to afford the acid (660 mg, 94%). ¹H NMR (300 MHz, CDCl₃) 11.5 (1H, br s), 8.26 (1H, br s), 7.48 (2H, m), 7.40 (2H, m), 7.26 (1H, m), 6.94 (1H, d), 2.87 92H, s), 1.97 (3H, br s), 1.63 (12H, m).

Step d. 5-[(2-Adamantan-1-ylmethyl-S-phenyl-1H-pyrrole-3-carbonyl)-amino]-isophthalic acid dibenzyl ester. To a suspension of the product of step c above (290 mg, 0.91 mmol) in DCM (5 ml) was added thionyl chloride (200 μl, 2.74 mmol) and one drop of DMF. The mixture was stirred at room temperature for 30 min, the solvent was evaporated and the residue was coevaporated with DCM (2×5 ml). 5-Amino-isophthalic acid dibenzyl ester (361 mg, 1.00 mmol) was added to the residue followed by anhydrous pyridine (2 ml). The solution was kept at room temperature for 16 h and diluted with DCM (30 ml). The organic phase was washed with 2M hydrochloric acid (2×20 ml), brine (20 ml), dried (MgSO₄) and the solvent was evaporated. The residue was purified by flash column chromatography (silica, DCM/hexane/ethyl acetate 9:9:2) to afford the product as pale yellow solid (300 mg, 45%). ¹H NMR (300 MHz, CDCl₃) 8.48 (3H, s), 8.30 (1H, br s), 7.69 (1H, s), 7.49-7.27 (15H, m), 6.66 (1H, d), 5.40 (4H, s), 2.91 (2H, s), 1.95 (3H, br s), 1.63 (12H, m).

Step e. A round bottom flask containing the product from the previous step (180 mg, 0.27 mmol), 10% palladium on charcoal (50 mg) and THF/methanol (1:1 mixture, 20 ml) was evacuated and flushed with hydrogen three times. The mixture was vigorously stirred overnight under an atmosphere of hydrogen. The catalyst was removed by filtration and the filtrate evaporated to afford the product as a white solid (130 mg, 98%). ¹H NMR (300 MHz, d₆-DMSO) 13.17 (2H, br s), 11.22 (1H, s), 9.80 (1H, s), 8.64 (2H, s), 8.13 (1H, s), 7.66 (2H, m), 7.39 (2H, m), 7.19 (2H, m), 2.85 (2H, s), 1.88 (3H, br s), 1.53 (12H, m). The acid was converted to the di(N-methyl-D-glucamine) salt and lyophilised from water/dioxan. Found: C 57.03, H 7.64, N, 6.03%; C₄₄H₆₄N₄O₁₅.2.0H₂O requires: C, 57.13; H, 7.41; N, 6.06%.

### EXAMPLE 20

5-[(5-Cycloheptylmethyl-2-naphthalen-2-yl-1H-imidazole-4-carbonyl)-amino]-isophtizalic Acid

Step a. 4-Cycloheptyl-2,3-dioxo-butyric acid ethyl ester monohydrate. Cycloheptaneacetic acid was converted to 4-cycloheptyl-2,3-dioxo-butyric acid ethyl ester hydrate according to the procedure of H. H. Wasserman (H. H. Wasserman, D. S. Ennis, C. A. Blum and V. M. Rotello, Tetrahedron Lett., 1992, 33, 6003). The tricarbonyl was isolated as pale yellow oil. ¹H NMR (300 MHz, CDCl₃) 4.99 (2H, br s), 4.30 (2H, q), 2.51 (2H, d), 2.14 (1H, m), 1.67-1.16 (15H, m).

Step b. 5-Cycloheptylmethyl-2-naphthalen-2-yl-1H-imidazole-4-carboxylic acid ethyl ester. This compound was prepared by modification of Brackeen's procedure (M. F. Brackeen, J. A. Stafford, P. L. Feldman and D. S. Karanewsky, Tetrahedron Lett., 1994, 35, 1635). To a slurry of ammonium acetate (9.0 g, 116 mmol) in acetic acid (35 ml) was added the product of step a (3.00 g, 11.6 mmol) followed by 2-naphthaldehyde (3.60 g, 23.2 mmol). The mixture was stirred in an oil bath heated to 70° C. for 2 h. The solution was cooled to room temperature and the acetic acid was evaporated. The residue was dissolved in ethyl acetate (50 ml) and washed with saturated sodium hydrogen carbonate (2×50 ml), water (20 ml) and brine (20 ml). The organic phase was dried (MgSO₄), the solvent was evaporated. The crude product was purified by crystallisation from ethyl acetate to afford the imidazole derivative as a white solid (1.74 g, 40%). ¹H NMR (300 MHz, CDCl₃) 10.18 and 9.97 (1H, br s), 8.39 (1H, s), 7.90 (4H, m), 7.52 (2H, br s), 4.40 (2H, q), 2.89 (2H, m), 2.05-1.26 (16H, m).

Step c. 5-Cycloheptylmethyl-2-naphthalen-2-yl-1H-imidazole-4-carboxylic acid. To a suspension of the product of step b (1.73 g, 4.62 mmol) in ethanol (25 ml) was added the solution of sodium hydroxide (1.29 g, 32.3 mmol) in water (5 ml). The reaction mixture was heated under reflux for 48 h, allowed to cool to room temperature and concentrated under reduced pressure. The aqueous solution was diluted with water (30 ml) and acidified to pH=2 with 1M hydrochloric acid. The precipitate was collected by filtration, washed with water, and dried to afford the product as an off-white solid (1.53 g, 96%). ¹H NMR (300 MHz, d₆-DMSO) 8.77 (1H, s), 8.24-7.98 (4H, m), 7.64 (2H, m), 2.91 and 2.64 (2H, 2×d), 2.03 (1H, m), 1.69-1.24 (12H, m).

Step d. S-[(5-Cycloheptylmethyl-2-naphthalen-2-yl-]H-imidazole-4-carbonyl)-amino]-isophthalic acid dibenzyl ester. To a solution of the product of step c above (500 mg, 1.44 mmol) and 5-amino-isophthalic acid dibenzyl ester (520 mg, 1.44 mmol) in DMF (3 ml) was added 1-hydroxybenzotriazole (HOBt) (195 mg, 1.44 mmol), 4-dimethylaminopyridine (DMAP) (cat.) and 1-(3-dimethylaminopropyl-3-ethylcarbodiimide hydrochloride (EDC) (280 mg, 1.44 mmol). The solution was kept at room temperature for 72 h, poured over 1M hydrochloric acid (20 ml) and the product was extracted with ethyl acetate (2×20 ml). The product crystallised from the ethyl acetate extracts. The crystals were collected by filtration, dried and triturated with methanol to afford a white solid (453 mg, 46%). ¹H NMR (300 MHz, d₆-DMSO) 13.00 (1H, br s), 10.50 (1H, s), 8.84 (2H, s), 8.61 (1H, s), 8.28 (2H, m), 8.00 (3H, m), 7.50 (12H, m), 5.41 (4H, s), 2.98 (2H, d), 2.00 (1H, m), 1.69-1.16 (12H, m).

Step e. The product of step d (450 mg, 0.65 mmol) was deprotected using the same procedure as in Example 1, step e to afford the title compound as a white solid (310 mg, 94%). ¹H NMR (300 MHz, d₆-DMSO) 10.75 (1H, s), 8.77 (3H, m), 8.40 (1H, d), 8.25 (1H, s), 8.13 (1H, d), 8.02 (2H, m), 7.65 (2H, m), 3.05 (2H, d), 2.11 (1H, m), 1.75-1.31 (12H, m). The acid was converted to the di(N-methyl-D-glucamine) salt and lyophilised from water/dioxan. Found: C, 53.27; H, 7.24; N, 7.09%; C₄₄H₆₃N₅O₁₅.4.8H₂O requires: C, 53.44; H, 7.41; N, 7.08%.

### EXAMPLE 36

5-[(1-Benzyl-5-cycloheptylmethyl-1H-imidazole-4-carbonyl)-amino]-isophthalic Acid

Step a. 1-Benzyl-5-cycloheptylmethyl-1H-imidazole-4-carboxylic acid ethyl ester. To a suspension of 5-cycloheptylmethyl-1H-imidazole-4-carboxylic acid ethyl ester (Example 35) (1.16 g, 4.63 mmol) in DMF (15 nm) was added sodium hydride (60% dispersion in oil) (200 mg, 5.10 mmol) in small portions. The resulting solution was stirred at room temperature for 1 h, then benzyl bromide (0.55 ml, 4.63 mmol) was added. The reaction mixture was stirred at room temperature for 2 h, the solvent was evaporated under reduced pressure and the residue was partitioned between saturated sodium hydrogen carbonate and ethyl acetate. The organic phase was washed with brine, dried (MgSO₄) and the solvent was evaporated. The residue was purified by flash column chromatography (silica, DCM/ethyl acetate 8:2). The major isomer (lower R_{f}) was isolated (855 mg, 54%). ¹H NMR (300 MHz,CDCl₃) 7.42 (1H, s), 7.34 (3H, m), 7.04 (2H, m), 5.09 (2H, s), 4.35 (2H, m), 2.81 (2H, d), 1.86-1.16 (16H, m).

Step b. 1-Benzyl-5-cycloheptylmethyl-1H-imidazole-4-carboxylic acid. To a solution of the product of step a (1.13 g, 3.32 mmol) in ethanol (20 ml) was added the solution of sodium hydroxide (1.33 g, 33.2 mmol) in water (5 ml). The solution was heated under reflux for 16 h, allowed to cool to room temperature, and concentrated under reduced pressure. The aqueous solution was diluted with water (20 ml), then acidified (pH=5.0, 1M HCl). The precipitate was collected by filtration, washed with water and dried to afford the acid as a white solid (1.03 g, 99%). ¹H NMR (300 MHz, d₆-DMSO) 7.62 (1H, s), 7.28 (3H, m), 7.05 (2H, m), 5.17 (2H, s), 2.77 (2H, d), 1.50 (5H, m), 1.37 (4H, m), 1.12 (4H, m).

Step c. 5-[(1-Benzyl-S-cycloheptylmethyl-1H-imidazole-4-carbonyl)-amino]-isophthalic acid dimethyl ester. The product of step b above (310 mg, 1.00 mmol) was reacted with 5-amino-isophthalic acid dimethyl ester (210 mg, 1.00 mmol) using essentially the same procedure as in Example 20, step d. The crude product was purified by flash column chromatography (silica, DCM/ethyl acetate 92:8) to afford colourless foam (110 mg, 20%). ¹H NMR (300 MHz,CDCl₃) 9.36 (1H, s), 8.53 (2H, s), 8.40 (1H, s), 7.35 (4H, m), 7.08 (2H, m), 5.12 (2H, s), 3.94 (6H, s), 1.90-1.22 (13H, m).

Step d. To a solution of the product of step c above (110 mg, 0.22 mmol) in 1:1 THF/methanol (2 ml) was added the solution of lithium hydroxide monohydrate (30 mg, 0.66 mmol) in water (1 ml). The solution was stirred at room temperature for 16 h, concentrated under reduced pressure, diluted with water (1 ml) and acidified (pH=2.0, 1M HCl). The precipitate was collected by filtration, washed with water and dried to afford the title compound as a white solid (92 mg, 89%). ¹H NMR (300 MHz, d₆-DMSO) 13.00 (2H, br s), 10.23 (1H, s), 8.64 (2H, s), 8.15 (1H, s), 7.92 (1H, s), 7.32 (3H, m), 7.14 (2H, m), 5.28 (2H, s), 2.83 (2H, d), 1.51 (5H, m), 1.37 (4H, m), 1.14 (4H, m). The acid was converted to the di(N-methyl-D-glucamine) salt and lyophilised from water/dioxan. Found: C, 54.47; H, 7.63; N, 7.59%; C₄₁H₆₃N₅O₁₅.2.2H₂O requires: C, 54.35; H, 7.50; N, 7.73%.

### EXAMPLE 44

5-{[5-(2-Adamantan-1-yl-ethyl)-2-naphthalen-2-yl-]H-imidazole-4-carbonyl]-amino}-isophtizalic Acid

5-Adamantan-1-yl-2,3-dioxo-pentanoic acid ethyl ester monohydrate was prepared from 3-(adamantan-1-yl)-propionic acid (W. Oppolzer and R. Moretti, Tetrahedron, 1988, 44, 5541) according to the procedure of Example 20, step a. It was then reacted with 2-naphthaldehyde and the ethyl ester was hydrolized to produce 5-(2-adamantan-1-yl-ethyl)-2-naphthalen-2-yl-1H-imidazole-4-carboxylic acid according to the procedure of Example 20, step b and c. This was converted to the title compound using the procedure of Example 20, steps d and e. ¹H NMR (300 MHz, d₆-DMSO) 8.72 (2H, s), 8.56 (1H, s), 8.30 (1H, d), 8.17 (1H, s), 8.02 (1H, d), 7.96 (2H, m), 7.56 (2H, m), 3.02 (2H, m), 1.98 (3H, br s), 1.68 (6H, m), 1.58 (6H, s), 1.45 (2H, m). The acid was converted to the di(N-methyl-D-glucamine) salt and lyophilised from water/dioxan. Found: C, 57.64; H, 7.62; N, 7.43%; C₄₈H₆₇N₅O₁₅.2.2H₂O requires: C 58.03H 7.24, N 7.05%.

### EXAMPLE 50

3-{[5-(2-Adamantan-1-yl-ethyl)-2-(2-fluoro-phenyl)-1H-imidazole-4-carbonyl]-amino}-benzoic Acid

5-Adamantan-1-yl-2,3-dioxo-pentanoic acid ethyl ester monohydrate (Example 44) was reacted with 2-fluorobenzaldehyde according to the procedure of Example 20, step b to produce 5-(2-adamantan-1-yl-ethyl)-2-(2-fluoro-phenyl)-1H-imidazole-4-carboxylic acid ethyl ester. The ester was hydrolyzed according to the procedure of Example 20, step c and the resulting 5-(2-adamantan-1-yl-ethyl)-2-(2-fluoro-phenyl)-1H-imidazole-4-carboxylic acid was reacted with 3-amino-benzoic acid benzyl ester using essentially the same procedure as in Example 20, step d. Deprotection was carried out according to the procedure of Example 20, step e to afford the title compound as a colourless foam. ¹H NMR (300 MHz, d₆-DMSO) 12.91(1H, br s), 12.53 (1H, s), 9.86 (1H, s), 8.51 (1H, s), 8.12 (1H, t), 8.00 (1H, d), 7.63 (1H, d), 7.40 (4H, m), 3.01 (2H, m), 1.95 (3H, s), 1.71-1.39 (14H, m). The acid was converted to the N-methyl-D-glucamine salt and lyophilized from water/dioxan. Found: C, 61.15; H, 6.96; N, 7.80%; C₃₆H₄₇FN₄O₈.1.3H₂O requires: C, 61.19; H, 7.08; N, 7.93%.

### EXAMPLE 60

3-{[5-(2-Adamantan-1-yl-ethyl)-2-(2,4-difluoro-phenyl)-1H-imidazole-4-carbonyl]-amino}-benzoic Acid

5-Adamantan-1-yl-2,3-dioxo-pentanoic acid ethyl ester monohydrate (Example 44) was reacted with 2,4-difluorobenzaldehyde according to the procedure of Example 20, step b to produce 5-(2-adamantan-1-yl-ethyl)-2-(2,4-difluoro-phenyl)-1H-imidazole-4-carboxylic acid ethyl ester. This was converted to the title compound using the procedure of Example 20, steps c, d and e with the modification that 3-amino-benzoic acid benzyl ester was used in step d instead of 5-amino-isophthalic acid dibenzyl ester. ¹H NMR (300 MHz, d₆-DMSO) 12.55 (1H, s), 9.85 (1H, s), 8.48 (1H, s), 8.13 (1H, m), 7.97 (1H, m), 7.62 (1H, m), 7.44 (2H, m), 7.25 (1H, m), 2.98 (2H, m), 1.95 (3H, s), 1.67 (6H, m), 1.54 (6H, s), 1.39 (2H, m). The acid was converted to the N-methyl-D-glucamine salt and lyophilised from water/dioxan. Found: C, 60.09; H, 6.77; N, 7.62%; C₃₆H₄₆F₂N₄O₈.1.1H₂O requires: C 59.96, H 6.74, N 7.77%.

### EXAMPLE 68

3-{[5-(2-Adamantan-1-yl-ethyl)-2-(3,4-dichloro-phenyl)-]H-imidazole-4-carbonyl]-amino}-benzoic Acid

5-Adamantan-1-yl-2,3-dioxo-pentanoic acid ethyl ester monohydrate (Example 44) was reacted with 3,4-dichlorobenzaldehyde according to the procedure of Example 20, step b to produce 5-(2-adamantan-1-yl-ethyl)-2-(3,4-dichloro-phenyl)-1H-imidazole-4-carboxylic acid ethyl ester. The ester was hydrolyzed according to the procedure of Example 20, step c and the resulting 5-(2-adamantan-1-yl-ethyl)-2-(3,4-dichloro-phenyl)-1H-imidazole-4-carboxylic acid was reacted with 3-amino-benzoic acid methyl ester using essentially the same procedure as in Example 52, step a. Deprotection was carried out following essentially the procedure of Example 36, step d to afford the title compound. ¹H NMR (300 MHz, d₆-DMSO) 12.89 (2H, br s), 9.90 (1H, s), 8.50 (1H, s), 8.32 (1H, s), 8.03 (2H, m), 7.76 (1H, d), 7.63 (1H, d), 7.43 (1H, t), 2.98 (2H, m), 1.97 (3H, s), 1.70-1.39 (14H, m). The acid was converted to the N-methyl-D-glucamine salt and lyophilised from water/dioxan. Found: C, 56.90; H, 6.44; N, 7.08%; C₃₆H₄₆Cl₂N₄O₀.1.6H₂O requires: C 56.72, H 6.50, N 7.35%.

### EXAMPLE 70

3-{[5-(2-Adamantan-1-yl-ethyl)-2-(2-dimethylamino-phenyl)-1H-imidazole-4-carbonyl]-amino}-benzoic Acid

Step a. 5-(2-Adamantan-1-yl-ethyl)-2-(2-dimethylamino-phenyl)-1H-imidazole-4-carboxylic acid benzyl ester. 5-Adamantan-1-yl-2,3-dioxo-pentanoic acid benzyl ester monohydrate (10.10 g, 3.00 mmol) (prepared from 3-(adamantan-1-yl)-propionic acid (W. Oppolzer and R. Moretti, Tetrahedron, 1988, 44, 5541) according to the procedure of Example 20, step a) was reacted with 2-dimethylamino-benzaldehyde (900 mg, 6.00 mmol) using essentially the procedure of Example 20, step b. The crude product was purified by flash column chromatography (silica, DCM/ethyl acetate 98:2) to afford the benzyl ester as a pale yellow oil (690 mg, 48%). ¹H NMR (300 MHz, CDCl₃) 12.25 and 12.10 (1H, 2×br s), 8.30 (1H, m), 7.35 (8H, m), 5.40 and 5.34 (2H, 2×s), 3.05 and 2.88 (2H, 2×m), 2.72 (6H, s), 1.93 (3H, br s), 1.65 (6H, m), 1.49 (8H, m).

Step b. 5-(2-Adamantan-1-yl-ethyl)-2-(2-dimethylamino-phenyl)-1H-imidazole-4-carboxylic acid. The product of step a (690 mg, 1.43 mmol) was deprotected using the same procedure as in Example 1, step e to afford the acid as a pale yellow solid (533 mg, 95%). ¹H NMR (300 MHz, CDCl₃) 8.29 (1H, d), 7.31 (3H, m), 3.02 (2H, m), 2.75 (6H, s), 1.98 (3H, br s), 1.69 (6H, m), 1.59 (6H, s), 1.48 (2H, m).

Step c. 3-{[5-(2-Adamantan-1-yl-ethyl)-2-(2-dimethylamino-phenyl)-1H-imidazole-4-carbonyl]-amino}-benzoic acid benzyl ester. The product of step b (310 mg, 0.80 mmol) was reacted with 3-amino-benzoic acid benzyl ester (220 mg, 0.95 mmol) according to the procedure given in Example 20, step d to afford a colourless foam after purification by flash column chromatography (silica, DCM/hexane/ethyl acetate 9:9:2) (278 mg, 58%). ¹H NMR (300M, CDCl₃) 12.10 (1H, br s), 9.33 (1H, s), 8.29 (2H, m), 8.17 (1H, s), 7.80 (1H, d), 7.49-7.26 (9H, m), 5.39 (2H, s), 3.14 (2H, m), 2.76 (6H, s), 2.00 (3H, br s), 1.76-1.60 (12H, m), 1.48 (2H, m).

Step d. The product of step c (278 mg, 0.46 mmol) was deprotected using the same procedure as in Example 1, step e to afford the title compound as a white solid (224 mg, 95%). ¹H NMR (300 MHz, d₆-DMSO) 13.00 (1H, br s), 10.00 (1H, br s), 8.47 (1H, s), 7.97 (2H, m), 7.64 (1H, d), 7.44 (3H, m), 7.25 (1H, m), 3.02 (2H, m), 2.76 (6H, br s), 1.94 (3H, s), 1.65 (6H, m), 1.54 (6H, s), 1.42 (2H, m). The acid was converted to the N-methyl-D-glucamine salt and lyophilised from water/dioxan. Found: C, 59.99; H, 7.65; N, 8.81%; C₃₈H₅₃N₅O₈.3.2H₂O requires: C, 59.69; H, 7.82; N, 9.16%.

### EXAMPLE 73

3-{[5-(2-Adamantan-1-yl-ethyl)-1-methyl-2-o-tolyl-1H-imidazole-4-carbonyl]-amino}-benzoic Acid

Step a. 5-(2-Adamantan-1-yl-ethyl)-1-methyl-2-o-tolyl-1H-imidazole-4-carboxylic acid ethyl ester and 5-(2-adamantan-1-yl-ethyl)-3-methyl-2-o-tolyl-3H-imidazole-4-carboxylic acid ethyl ester. To a solution of 5-(2-adamantan-1-yl-ethyl)-2-o-tolyl-1H-imidazole-4-carboxylic acid ethyl ester (Example 57) (784 mg, 2.00 mmol) in DMF (7.5 ml) was added sodium hydride (60% dispersion in oil) (100 mg, 2.50 mmol) in small portions at 0° C. The mixture was stirred at room temperature for 30 min, iodomethane (140 μl, 2.20 mmol) was added and the stirring was continued for 2 h. The reaction mixture was partitioned between diethyl ether and 1M sodium hydroxide, the organic phase was washed with water, dried and the solvent was evaporated. The products were separated by flash column chromatography (silica, DCM/ethyl acetate 9:1, then 1:1). The high R_{f} product (257 mg, 32%) was identified as 5-(2-adamantan-1-yl-ethyl)-3-methyl-2-o-tolyl-3H-imidazole-4-carboxylic acid ethyl ester, whilst the low R_{f} material (397 mg, 49%) was the isomeric 5-(2-adamantan-1-yl-ethyl)-1-methyl-2-o-tolyl-1H-imidazole-4-carboxylic acid ethyl ester. ¹H NMR (300 MHz,CDCl₃) of the high R_{f} material 7.34-7.24 (4H, m), 4.36 (2H, q), 3.62 (3H, s), 2.89 (2H, m), 2.17 (3H, s), 1.96 (3H, s), 1.74-1.57 (12H, m), 1.48 (2H, m), 1.42 (3H, t). ¹H NMR (300 MHz,CDCl₃) of the low R_{f} material 7.33-7.22 (4H, m), 4.36 (2H, q), 3.31 (3H, s), 2.98 (2H, m), 2.15 (3H, s), 1.99 (3H, s), 1.75-1.60 (12H, m), 1.34 (5H, m).

Step b. 5-(2-Adamantan-1-yl-ethyl)-1-methyl-2-o-tolyl-1H-imidazole-4-carboxylic acid. To a solution of 5-(2-adamantan-1-yl-ethyl)-1-methyl-2-o-tolyl-1H-imidazole-4-carboxylic acid ethyl ester (397 mg, 0.98 mmol) in ethanol (30 ml) was added 2.0M potassium hydroxide solution (3.4 ml). The solution was heated at reflux for 16 h, cooled to 50° C. and acidified (1M hydrochloric acid, pH=34). The ethanol was evaporated, the residue was diluted with water (30 ml) and the product was extracted with DCM. The organic layer was dried (MgSO₄) and the solvent was evaporated to afford the product as a white solid (351 mg, 95%). ¹H NMR (300 MHz,CDCl₃) 7.41-7.27 (4H, m), 6.00 (1H, br s), 3.38 (3H, s), 3.01 (2H, m), 2.20 (3H, s), 2.01 (3H, s), 1.70 (6H, m), 1.61 (6H, s), 1.37 (2H, m).

Step c. 3-{[5-(2-Adamantan-1-yl-ethyl)-1-methyl-2-o-tolyl-1H-imidazole-4-carbonyl]-amino}-benzoic acid benzyl ester. The product of step b above (351 mg, 0.93 mmol) was reacted with 3-amino-benzoic acid benzyl ester (211 mg, 0.93 mmol) using essentially the same procedure as in Example 70, step c to afford the benzyl ester as a white solid (388 mg, 71%). ¹H NMR (300 MHz,CDCl₃) 9.19 (1H, s), 8.15 (1H, d), 8.13 (1H, s), 7.78 (1H, d), 7.45-7.31 (9H, m), 5.36 (2H, s), 3.37 (3H, s), 3.11 (2H, m), 2.23 (3H, s), 2.02 (3H, s), 1.77-1.64 (12H, m), 1.30 (2H, m).

Step d. The product of step c (388 mg, 0.66 mmol) was deprotected using the same procedure as in Example 1, step e to afford the title compound as a white solid (292 mg, 89%). ¹H NMR (300 MHz, d₆-DMSO) 12.50 (1H, br s), 9.85 (1H, s), 8.53 (1H, s), 7.92 (1H, d), 7.60 (1H, m), 7.36 (5H, m), 3.35 (3H, s), 3.02 (2H, m), 2.16 (3H, s), 1.96 (3H, s), 1.71-1.58 (12H, m), 1.32 (2H, m). The acid was converted to the N-methyl-D-glucamine salt and lyophilised from water/dioxan. Found: C, 63.42; H, 8.00; N, 7.75%; C₃₈H₅₂N₄O₈.1.5H₂O requires: C, 63.40; H, 7.70; N, 7.78%.

### EXAMPLE 74

3-{[5-(2-Adamantan-1-yl-ethyl)-3-methyl-2-o-tolyl-3H-imidazole-4-carbonyl]-amino}-benzoic Acid

5-(2-Adamantan-1-yl-ethyl)-3-methyl-2-o-tolyl-3H-imidazole-4-carboxylic acid ethyl ester (Example 73, step a) was converted to the title compound according to the procedure of Example 73, steps b, c and d. ¹H NMR (300 MHz, d₆-DMSO) 13.00 (1H, br s), 9.84 (1H, s), 8.52 (1H, m), 7.91 (1H, m), 7.60 (1H, d), 7.36 (5H, m), 3.34 (3H, s), 3.03 (2H, m), 2.16 (3H, s), 1.96 (3H, s), 1.71-1.58 (12H, m), 1.32 (2H, m).

### EXAMPLE 753

3-[3-[5-(2-Adamantan-1-yl-ethyl)-2-o-tolyl-1H-imidazole-4-yl]-ureido}-benzoic Acid

Step a. 5-(2-Adamantan-1-yl-ethyl)-2-o-tolyl-]H-imidazole-4-carboxylic acid benzyl ester. 5-Adamantan-1-yl-2,3-dioxo-pentanoic acid benzyl ester monohydrate (8.44 g, 22.8 mmol) (prepared from 3-(adamantan-1-yl)-propionic acid (W. Oppolzer and R. Moretti, Tetrahedron, 1988, 44, 5541) according to the procedure of Example 20, step a) was reacted with o-tolualdehyde (5.47 g, 44.0 mmol) using essentially the procedure of Example 20, step b. The crude product was purified by flash column chromatography (silica, hexane/DCM/ethyl acetate 9:9:2) to afford the benzyl ester as a colourless foam (5.54 g, 54%). ¹H NMR (300 MHz, CDCl₃) 9.71 (1H, br s), 7.63 (1H, d), 7.49-7.27 (8H, m), 5.36 (2H, s), 2.92 (2H, m), 2.58 (3H, s), 1.97 (3H, br s), 1.67 (6H, m), 1.48 (8H, m).

Step b. 5-(2-Adamantan-1-yl-ethyl)-2-o-tolyl-imidazole-1,4-dicarboxylic acid 4-benzyl ester 1-tert-butyl ester. To a solution of the product of step a above (454 mg, 1.00 mmol) in anhydrous dioxan (5 ml) was added di-tert-butyl dicarbonate (264 mg, 1.20 mmol) and 4-dimethylaminopyridine (20 mg). The solution was stirred at room temperature for 3 h, then the solvent was evaporated. The crude product was purified by flash column chromatography (silica, hexane/DCM/ethyl acetate 5:4:1). The major isomer (300 mg, 54%) (low R_{f}) was used in step c. ¹H NMR (300 MHz, CDCl₃) 7.51 (2H, d), 7.43-7.24 (8H, m), 5.44 (2H, s), 3.25 (2H, m), 2.23 (3H, s), 1.99 (3H, br s), 1.70 (6H, m), 1.52 (6H, s), 1.35 (2H, m), 1.22 (9H, s).

Step c. 5-(2-Adamantan-]-yl-ethyl)-2-o-tolyl-imidazole-1,4-dicarboxylic acid 1-tert-butyl ester. The benzyl ester of the product of step b (300 mg, 0.54 mmol) was hydrogenolyzed using the same procedure as in Example 1, step e to afford the acid as a colourless foam (237 mg, 95%). ¹H NMR (300 MHz, CDCl₃) 7.40 (1H, m), 7.30 (4H, m), 3.32 (2H, m), 2.24 (3H, s), 2.04 (3H, br s), 1.80-1.63 (12H, m), 1.44 (2H, m), 1.25 (9H, s).

Step d. 5-(2-Adamantan-1-yl-ethyl)-4-[3-(3-benzyloxycarbonyl-phenyl)-ureido]-2-o-tolyl-imidazole-1-carboxylic acid tert=butyl ester. To a solution of the product of step c above (464 mg, 1.00 mmol) and triethylamine (186 μl, 1.33 mmol) in anhydrous acetone (7 ml) was slowly added a solution of ethyl-chloroformate (186 ml, 2.00 mmol) in anhydrous acetone (2 ml) at 0° C. The reaction mixture was stirred at 0° c for 30 min, then a solution of sodium azide (100 mg, 1.50 mmol) in water (1 ml) was added dropwise and the stirring was continued at room temperature for 30 min. The reaction mixture was diluted with water (20 ml) and the acetone was evaporated under reduced pressure. The aqueous layer was extracted with toluene (2×10 ml), and the combined toluene layers were dried (MgSO₄) and filtered. The filtrate was heated at reflux for 1 h, then 3-amino-benzoic acid benzyl ester was added and the mixture was heated at reflux for further 2 h. The reaction mixture was cooled; the solvent was evaporated in vacuo. The crude product was purified by flash column chromatography (silica, DCM/ethyl acetate 9:1) to afford the product as a white solid (237 mg, 34%). ¹H NMR (300 MHz, CDCl₃) 10.24 (1H, s), 8.03 (1H, s), 7.83 (1H, d), 7.74 (1H, d), 7.44-7.24 (10H, m), 6.48 (1H, s), 5.35 (2H, s), 2.77 (2H, m), 2.24 (3H, s), 1.98 (3H, br s), 1.65 (6H, m), 1.55 (6H, s), 1.29 (2H, m), 1.20 (9H, s).

Step e. 3-{3-[5-(2-Adamantan-1-yl-ethyl)-2-o-tolyl-1H-imidazol-4-yl]-ureido}-benzoic acid benzyl ester. The product of step d (237 mg, 0.34 mmol) was dissolved in trifluoroacetic acid (2 ml) and the solution was stirred at room temperature for 1 h. The trifluroracetic acid was removed in vacuo. The residue was partitioned between DCM and saturated aqueous sodium hydrogen carbonate. The organic layer was dried (MgSO₄) and the solvent was evaporated to afford the product as a white solid (190 mg, 94%). ¹H NMR (300 MHz, CDCl₃) 10.84 (1H, br s), 8.96 (1H, br s), 8.08 (1H, s), 7.75 (1H, d), 7.52 (1H, d), 7.47-7.27 (10H, m), 6.86 (1H, s), 5.36 (2H, s), 2.65 (3H, s), 2.57 (2H, m), 1.95 (3H, br s), 1.65 (6H, m), 1.51 (6H, s), 1.37 (2H, m).

Step f. The product of step e (190 mg, 0.32 mmol) was deprotected using the procedure of Example 1, step e to afford the title compound as a white solid. ¹H NMR (300 MHz, d₆-DMSO) 12.40 (1H, br s), 12.0 (1H, br s), 10.0(1H, br s), 8.20 (1H, br s), 8.11 (1H, s), 7.67-7.25 (7H, m), 2.59 (3H, s), 2.50 (2H, m), 1.92 (3H, s), 1.76-1.58 (12H, m), 1.38 (2H, m). The acid was converted to the N-methyl-D-glucamine salt and lyophilised from water/dioxan. Found: C, 60.21; H, 7.83; N, 9.26%; C₃₇H₅₁N₅O₈.1.5H₂O requires: C 60.01H 7.65, N 9.46%.

### EXAMPLE 92

N-[5-(2-Adamantan-1-yl-ethyl)-2-o-tolyl-1H-imidazol-4-yl]-isophthalamic Acid

Step a. 5-(2-Adamantan-1-yl-ethyl)-4-benzyloxycarbonylamino-2-o-tolyl-imidazole-1-carboxylic acid tert-butyl ester. 5-(2-Adamantan-1-yl-ethyl)-2-o-tolyl-imidazole-1,4-dicarboxylic acid 1-tert-butyl ester (Example 75, step c) was converted to the isocyanate and reacted with benzyl alcohol according to the procedure of Example 75, step d using benzyl alcohol in place of 3-amino-benzoic acid benzyl ester. The crude reaction mixture was purified by flash column chromatography (silica, hexane/ethyl acetate 3:2) to give the product. ¹H NMR (300 MHz, CDCl₃) 7.39-7.19 (9H, m), 6.10 (1H, br s), 5.20 (2H, s), 2.79 (2H, m), 2.18 (3H, s), 1.96 (3H, s), 1.68 (6H, m), 1.50 (6H, s), 1.31 (2H, m), 1.19 (9H, s).

Step b. 5-(2-Adamantan-1-yl-ethyl)-4-amino-2-o-tolyl-imidazole-1-carboxylic acid tert-butyl ester. The product from the previous step (340 mg, 0.57 mmol) was hydrogenolyzed using the same procedure as in Example 1, step e to afford the amine (175 mg, 71%). ¹H NMR (300 MHz, CDCl₃) 7.25-7.18 (4H, m), 3.28 (2H, br s), 2.72 (2H, m), 2.18 (3H, s), 1.98 (3H, s), 1.69 (6H, m), 1.57 (6H, m), 1.32 (211, m), 1.18 (9H, s).

Step C. 5-(2-Adamantan-1-yl-ethyl)-4-(3-methoxycarbonyl-benzoylamino)-2-o-tolyl-imidazole-1-carboxylic acid tert-butyl ester. Isophthalic acid mono methyl ester (200 mg, 1.10 mmol) was heated at reflux for 15 min in the mixture of thionyl chloride (2 ml) and DMF (cat.). The solvent was evaporated in vacuo, the residue was dissolved in DCM (5 ml) and the solvent was evaporated to afford 3-chlorocarbonyl-benzoic acid methyl ester. To a solution of the product of step b above (435 mg, 1.00 mmol) and triethylamine (280 μl, 2.00 mmol) in DCM (10 ml) was added a solution of the previously prepared 3-chlorocarbonyl-benzoic acid methyl ester in DCM (2 ml). The reaction mixture was stirred at room temperature for 1 h. It was washed with 5% aqueous potassium hydrogen sulfate (10 ml) and water (10 ml), dried (MgSO₄) and the solvent was evaporated. The residue was purified by flash column chromatography (silica, DCM/ethyl acetate 4:1) to afford the product as a white solid (310 mg, 52%). ¹H NMR (300 MHz, CDCl₃) 8.53 (1H, s), 8.18 (2H, m), 8.07 (1H, d), 7.51 (1H, t), 7.30-7.21 (4H, m), 3.95 (3H, s), 2.91 (2H, m), 2.20 (3H, s), 1.94 (3H, br s), 1.66 (6H, m), 1.52 (6H, s), 1.38 (2H, m), 1.21 (9H, s).

Step d. N-[5-(2-Adamantan-1-yl-ethyl)-2-o-tolyl-1H-imidazol-4-yl]-isophthalamic acid methyl ester. The product of step c above was deprotected according to the procedure of Example 75, step e. ¹H NMR (300 MHz, d₆-DMSO) 11.95 (1H, s), 10.12 (1H, s), 8.55 (1H, s), 8.24 (1H, d), 8.13 (1H, d), 7.66 (1H, t), 7.58 (1H, m), 7.26 (3H, m), 3.89 (3H, s), 2.54 (3H, s), 2.50 (2H, m), 1.89 (3H, br s), 1.63 (6H, m), 1.42 (8H, m).

Step e. The methyl ester was hydrolyzed following essentially the procedure of Example 36, step d to afford the title compound as a beige solid. ¹H NMR (300 MHz,d₆-DMSO) 13.00 (1H, br s), 11.96 (1H, br s), 10.09 (1H, s), 8.53 (1H, s), 8.20 (1H, d), 8.11 (1H, d), 7.62 (2H, m), 7.26 (3H, m), 2.54 (3H, s), 2.50 (2H, m), 1.89 (3H, br s), 1.62 (6H, m), 1.42 (8H, m). The acid was converted to the N-methyl-D-glucamine salt and lyophilised from water/dioxan. Found: C, 61.34; H, 7.72; N, 7.57%; C₃₇H₅₀N₄O₈.2.5H₂O requires: C, 61.39; H, 7.66; N, 7.74%.

### EXAMPLE 93

(±)-1-[5-(2-Adamantan-1-yl-ethyl)-2-o-tolyl-1H-imidazole-4-carbonyl]-piperidine-3-carboxylic Acid

5-(2-Adamantan-1-yl-ethyl)-2-o-tolyl-1H-imidazole-4-carboxylic acid was prepared according to the procedure of Example 70, steps a and b, with the modification that o-tolualdehyde was used in step a instead of 2-dimethylamino-benzaldehyde. The acid was converted to the title compound according to the procedure of Example 70, steps c and d, using (+)-piperidine-3-carboxylic acid benzyl ester in step c instead of 3-amino-benzoic acid benzyl ester. ¹H NMR (300 MHz, d₆-DMSO) 12.30 (2H, br s), 7.59 (1H, m), 7.26 (3H, m), 5.20-4.30 (2H, br m), 3.60-3.00 (2H, br m), 2.77 (2H, in), 2.51 (3H, s), 2.48 (1H, m), 2.00-1.35 (21H, m). The acid was converted to the N-methyl-D-glucamine salt and lyophilised from water/dioxan. Found: C, 58.45; H, 7.75; N, 7.74%; C₃₆H₅₄N₄O₈.1.0 DCM requires: C, 58.75; H, 7.46; N, 7.40%.

### EXAMPLE 94

(±)-]-[5-(2-Adamantan-1-yl-ethyl)-2-o-tolyl-1H-imidazole-4-carbonyl]-piperidine-4-carboxylic Acid

5-(2-Adamantan-1-yl-ethyl)-2-o-tolyl-1H-imidazole-4-carboxylic acid (Example 93) was converted to the title compound according to the procedure of Example 70, steps c and d, using (+)-piperidine-4-carboxylic acid benzyl ester in step c instead of 3-amino-benzoic acid benzyl ester. ¹H NMR (300 MHz, d-DMSO) 12.29 (1H, br s), 12.19 (1H, s), 7.58 (1H, m), 7.26 (3H, m), 5.20-4.30 (2H, br m), 3.40-2.80 (2H, br m), 2.75 (2H, m), 2.52 (3H, s), 1.93-1.35 (22H, m). The acid was converted to the N-methyl-D-glucamine salt and lyophilised from water/dioxan. Found: C, 61.00; H, 8.33; N, 7.98%; C₃₆H₅₄N₄O₈.2.0H₂O requires: C, 61.17; H, 8.27; N, 7.92%.

### EXAMPLE 102

{[5-(2-Adamantan-1-yl-ethyl)-2-o-tolyl-]H-imidazole-4-carbonyl]-amino}-phenyl-acetic Acid

Step a. {[5-(2-Adamantan-1-yl-ethyl)-2-o-tolyl-1H-imidazole-4-carbonyl]-amino}-phenyl-acetic acid methyl ester. To a solution of 5-(2-adamantan-1-yl-ethyl)-2-o-tolyl-1H-imidazole-4-carboxylic acid (Example 93) (389 mg, 1.07 mmol) and DL-α-phenylglycine methyl ester hydrochloride (258 mg, 1.28 mmol) in DMF (5 ml) was added triethylamine (196 μl, 1.41 mmol), 1-hydroxybenzotriazole (203 mg, 1.50 mmol) and 1-(3-dimethylaminopropyl-3-ethylcarbodiimide hydrochloride (288 mg, 1.50 mmol). The solution was kept at room temperature for 72 h, poured into 1M hydrochloric acid (20 ml) and the product was extracted with ethyl acetate (2×20 ml). The solvent was evaporated and the residue was purified by flash column chromatography (silica, hexane/ethyl acetate 2:1) to afford the product as a colourless foam (447 mg, 82%). ¹H NMR (300 MHz, CDCl₃) 9.11 (1H, br s),8.13 (1H, br d), 7.55-7.26 (9H, m), 5.80 (1H, d), 3.75 (3H, s), 3.07 (2H, m), 2.57 (3H, s), 1.96 (3H, s), 1.73-1.54 (12H, m), 1.42 (2H, m).

Step b. To a solution of the product of step a above (441 mg, 0.86 mmol) in water (4 ml) and THF (4 ml) was added lithium hydroxide monohydrate (181 mg, 4.31 mmol). The solution was stirred at room temperature for 16 h, acidified with 2M hydrochloric acid (pH=1), diluted with water (20 ml). The product was extracted with ethyl acetate (2×30 ml), the organic phase was dried (MgSO₄) and the solvent was evaporated to afford the title compound as a white solid (391 mg, 91%). ¹H NMR (300 MHz, d₆-DMSO) 13.00 (1H, br s), 12.42 (1H, br s), 8.11 (1H, d), 7.56 (1H, m), 7.44-7.27 (8H, m), 5.51 (1H, d), 2.90 (2H, m), 2.51 (3H, s), 1.91 (3H, br s), 1.63 (6H, m), 1.49 (6H, s), 1.37 (2H, m). The acid was converted to the N-methyl-D-glucamine salt and lyophilised from water/dioxan. Found: C 63.87, H 7.64, N 7.92%; C₃₈H₅₂N₄O₈.1.0H₂O requires: C 64.21, H 7.66, N 7.88%.

### EXAMPLE 120

3-{[5-(2-Adamantan-1-yl-ethyl)-2-O-tolyl-oxazole-4-carbonyl]-amino}-benzoic Acid

Step a. 5-Adamantan-1-yl-2-(2-methyl-benzoylamino)-3-oxo-pentanoic acid ethyl ester. 5-Adamantan-1-yl-2-amino-3-oxo-pentanoic acid ethyl ester hydrochloride (prepared using the method of T. W. von Geldern & C. Hutchins et al, J. Med. Chem. 1996, 39, 957) (3.30 g, 10.0 mmol) was suspended in DMF (15 ml) and a solution of 2-methylbenzoyl chloride (1.55 g 10.0 mmol) in THF (15 ml) was added. The mixture was stirred at room temperature and a solution of N,N-diispropylethylamine (3.48 ml 20.0 mmol) in THF (10 ml) was added dropwise over 30 min. After stirring for 2 h the THF was evaporated and the residue was partitioned between ethyl acetate and saturated sodium bicarbonate. The organic layer was separated, washed with water, 2M HCl, brine, dried (MgSO₄), and the solvent was evaporated. The residue was purified by flash column chromatography (silica, DCM/hexane/ethyl acetate 9:9:2) to afford the product as a pale yellow oil. (2.08 g, 60%). ¹NMR (300 MHz, CDCl₃) 7.49-7.23 (4H, m), 6.97 (1H, d), 5.45 (1H, d), 4.30 (2H, m), 2.77 (2H, m), 2.47 (3H, s), 1.97 (3H, s), 1.67 (6H, m), 1.47 (8H, m), 1.34 (3H, t).

Step b. 5-(2-Adamantan-1-yl-ethyl)-2-o-tolyl-oxazole-4-carboxylic acid ethyl ester. To a solution of the product from step a (1.00 g, 2.43 mmol) in acetonitrile (4 ml) and pyridine (4 ml) was added sequentially carbon tetrachloride (486 μl, 4.88 mmol) 1,8-diazabicyclo[5,4,0]undec-7-ene (1.22 ml, 8.13 mmol), and triphenylphosphine (693 mg, 2.64 mmol). The mixture was left to stand at room temperature for 16 h. The solvent was evaporated and the residue was dissolved in DCM. The solution was washed with saturated sodium bicarbonate, brine, 1M phosphoric acid, brine, dried (MgSO₄), and the solvent was evaporated. The residue was purified by flash column chromatography (silica, DCM/hexane/ethyl acetate 9:9:2) to afford the product as a pale yellow oil (540 mg, 57%). ¹H NMR (300 MHz, CDCl₃) 7.98 (1H, d), 7.32 (3H, m), 4.42 (2H, q), 3.08 (2H, m), 2.68 (3H, s), 2.00 (3H s), 1.76-1.40 (14H, m), 1.26 (3H, t).

Step c 5-(2-Adamantan-1-yl-ethyl)-2-o-tolyl-oxazole-4-carboxylic acid. A solution of the product from step b (540 mg, 1.37 mmol) in ethanol (48 ml) and 2M sodium hydroxide (4.8 ml, 9.60 mmol) were refluxed for 16 h. The resulting hot suspension was acidified (pH=3, 1M H₃PO₄), the ethanol was evaporated, the residue was diluted with water and extracted with ethyl acetate. The organic layer was washed with water, dried (MgSO₄), and the solvent was evaporated to afford the product as a white solid (460 mg, 92%). ¹H NMR (300 MHz, CDCl₃) 8.00 (1H, d), 7.33 (3H, m), 3.11 (2H, m), 2.70 (3H, s), 2.00 (3H, s), 1.77-1.49 (14H, m).

Step d. 3-{[5-(2-Adamantan-1-yl-ethyl)-2-o-tolyl-oxazole-4-carbonyl]-amino}-benzoic acid benzyl ester. The acid from step c was coupled to 3-amino-benzoic acid benzyl ester using essentially the procedure of Example 52, step a to afford the product as a white solid (328 mg, 45%). ¹H NMR (300 MHz, CDCl₃) 8.96 (1H, s), 8.20 (1H, d), 8.14 (1H, s), 7.98 (1H, d), 7.83 (1H, d), 7.49-7.33 (9H, m), 5.4 (2H, s), 3.19 (2H, m), 2.73 (3H, s), 2.00 (3H, s), 1.76-1.54 (14H, m).

Step e. The product of step d (328 mg, 0.57 mmol) was deprotected using the same procedure as in Example 1, step e to afford the title compound as a white solid (273 mg, 99%). ¹H NMR (300 MHz, d₆-DMSO) 12.96 (1H, s), 10.04 (1H, s), 8.48 (1H, s), 7.99 (2H, m), 7.68 (1H, d), 7.49-7.37 (4H, m), 3.12 (2H, m), 2.68 (3H, s), 1.94 (3H, s), 1.75-1.46 (14H, m). The acid was converted to the N-methyl-D-glucamine salt and lyophilised from water/dioxan. Found: C, 63.50; H, 7.46; N, 5.96%. C₃₇H₄₉N₃O₉.H₂O requires: C 63.68, H 7.37, N 6.02%.Step e. The product of step d (328 mg, 0.57 mmol) was deprotected using the same procedure as in Example 1, step e to afford the title compound as a white solid (273 mg, 99%). ¹H NMR (300 MHz, d₆-DMSO) 12.96 (1H, s), 10.04 (1H, s), 8.48 (1H, s), 7.99 (2H, m), 7.68 (1H, d), 7.49-7.37 (4H, m), 3.12 (2H, m), 2.68 (3H, s), 1.94 (3H, s), 1.75-1.46 (14H, m). The acid was converted to the N-methyl-D-glucamine salt and lyophilised from water/dioxan. Found: C, 63.50; H, 7.46; N, 5.96%. C₃₇H₄₉N₃O₉.H₂O requires: C, 63.68; H, 7.37; N, 6.02%.Step e. The product of step d (328 mg, 0.57 mmol) was deprotected using the same procedure as in Example 1, step e to afford the title compound as a white solid (273 mg, 99%). ¹H NMR (300 MHz, d₆-DMSO) 12.96 (1H, s), 10.04 (1H, s), 8.48 (1H, s), 7.99 (2H, m), 7.68 (1H, d), 7.49-7.37 (4H, m), 3.12 (2H, m), 2.68 (3H, s), 1.94 (3H, s), 1.75-1.46 (14H, m). The acid was converted to the N-methyl-D-glucamine salt and lyophilised from water/dioxan. Found: C, 63.50; H, 7.46; N, 5.96%. C₃₇H₄₉N₃O₉.H₂O requires: C, 63.68; H, 7.37; N, 6.02%.

### EXAMPLE 121

3-{[5-(2-Adamantan-1-yl-ethyl)-2-o-tolyl-thiazole-4-carbonyl]-amino}-benzoic Acid.

Step a. 5-(2-Adamantan-1-yl-ethyl)-2-o-tolyl-thiazole-4-carboxylic acid ethyl ester. A solution of the product from Example 120, step a (1.00 g, 2.43 mmol) and Lawesson's reagent (1.23 g, 3.00 mmol) in THF (16 ml) was refluxed for 4 hrs. The solvent was evaporated and the residue was taken up in DCM. The solution was washed with saturated sodium bicarbonate, brine, 1M phosphoric acid, then dried (MgSO₄), and the solvent was evaporated. The residue was purified by flash column chromatography (silica, DCM/hexane/ethyl acetate 9:9:2) to afford the product as a white solid (400 mg, 40%). ¹H NMR (300 MHz, CDCl₃) 7.64 (2H, d), 7.29 (3H, m), 4.44 (2H, q), 3.26 (2H, m), 2.57 (3H, s), 2.01 (3H, s), 1.77-1.49 (14H, m), 1.43 (3H, t). A solution of the product from Example 120, step a (1.00 g, 2.43 mmol) and Lawesson's reagent (1.23 g, 3.00 mmol) in THF (16 ml) was refluxed for 4 hrs. The solvent was evaporated and the residue was taken up in DCM. The solution was washed with saturated sodium bicarbonate, brine, 1M phosphoric acid, then dried (MgSO₄), and the solvent was evaporated. The residue was purified by flash column chromatography (silica, DCM/hexane/ethyl acetate 9:9:2) to afford the product as a white solid (400 mg, 40%). ¹H NMR (300 MHz, CDCl₃) 7.64 (2H, d), 7.29 (3H, m), 4.44 (2H, q), 3.26 (2H, m), 2.57 (3H, s), 2.01 (3H, s), 1.77-1.49 (14H, m), 1.43 (3H, t). A solution of the product from Example 120, step a (1.00 g, 2.43 mmol) and Lawesson's reagent (1.23 g, 3.00 mmol) in TH-F (16 ml) was refluxed for 4 hrs. The solvent was evaporated and the residue was taken up in DCM. The solution was washed with saturated sodium bicarbonate, brine, 1M phosphoric acid, then dried (MgSO₄), and the solvent was evaporated. The residue was purified by flash column chromatography (silica, DCM/hexane/ethyl acetate 9:9:2) to afford the product as a white solid (400 mg, 40%). ¹H NMR (300 MHz, CDCl₃) 7.64 (2H, d), 7.29 (3H, m), 4.44 (2H, q), 3.26 (2H, m), 2.57 (3H, s), 2.01 (3H, s), 1.77-1.49 (14H, m), 1.43 (3H, t).

Step b. 5-(2-Adamantan-1-yl-ethyl)-2-o-tolyl-thiazole-4-carboxylic acid. The ethyl ester from step a (400 mg, 0.98 mmol) was hydrolysed using essentially the same procedure as in Example 120, step c to afford the product as a white solid (351 mg, 94%). ¹H NMR (300 MHz, CDCl₃) 7.65 (1H, d), 7.35 (3H, m), 3.31 (2H, m), 2.60 (3H, s), 2.00 (3H, s), 1.76-1.50 (14H, m).

Step c. 3-{[5-(2-Adamantan-1-yl-ethyl)-2-o-tolyl-thiazole-4-carbonyl]-amino}-benzoic acid methyl ester. The acid from step b was reacted with 3-amino-benzoic acid methyl ester using essentially the same procedure as in Example 52, step a to afford the product as a white solid. ¹H NMR (300 MHz, CDCl₃) 9.55 (1H, s), 8.17 (1H, s), 8.11 (1H, d), 7.79 (1H, d), 7.70 (1H, d), 7.48-7.32 (4H, m), 3.94 (3H, s), 3.43 (2H, m), 2.65 (3H, s), 2.01 (3H, s), 1.73-1.54 (14H, m).

Step d. The product of step c (351 mg, 0.68 mmol) was hydrolysed using essentially the same procedure as in Example 118, step d, to afford the title compound as a white solid (315 mg, 93%). ¹H NMR (300 MHz, d₆-DMSO) 12.94 (1H, s), 10.24 (1H, s), 8.46 (1H, s), 8.00 (1H, d), 7.90 (1H, d), 7.68 (1H, d), 7.49-7.34 (4H, m), 3.34 (2H, m), 2.60 (3H, s), 1.95 (3H, s), 1.70-1.45 (14H, m). The acid was converted to the N-methyl-D-glucamine salt and lyophilised from water/dioxan. Found: C, 61.40; H, 7.52; N, 5.85%. C₃₇H₄₉N₃O₈S.1.5H₂O requires: C, 61.47; H, 7.25; N, 5.81%.

### EXAMPLE 122

3-{[4-(2-Adamantan-1-yl-ethyl)-2-cyclohexyl-thiazole-5-carbonyl]-amino}-benzoic Acid.

Step a. 4-Adamantan-1-yl-2-chloro-3-oxo-butyric acid ethyl ester. To a solution of 4-adamantan-1-yl-3-oxo-butyric acid ethyl ester (prepared by the method of W. Wierenga & H. I. Skulnick, J. Org. Chem. 1979, 44, 310) (2.78 g, 10.0 mmol) in DCM (10 ml) was added dropwise a solution of sulfuryl chloride (0.843 ml, 10.5 mmol) in DCM (10 ml) over 10 min. The solution was stirred at room temperature for 1 h, then it was washed with water, brine, dried (MgSO₄), and the solvent was evaporated to afford the product as an orange oil (2.39 g, 73%). ¹H NMR (300 MHz, CDCl₃) 5.30 (1H, s), 4.27 (2H, m), 2.65 (2H, m), 1.96 (3H, s), 1.74-1.32 (14H, m), 1.30 (3H, t).

Step b. 4-(2-Adamantan-1-yl-ethyl)-2-cyclohexyl-thiazole-5-carboxylic acid ethyl ester. A solution of the product from step a (2.39 g, 7.65 mmol) and cyclohexanethiocarboxamide (1.19 g 7.65 mmol) in ethanol (30 ml) was refluxed for 2 h. The solvent was evaporated and the residue was taken up in ether. The solution was washed with saturated sodium bicarbonate, brine, dried (MgSO₄), and the solvent was evaporated. The residue was purified by flash column chromatography (silica, hexane/ethyl acetate 9:1) to afford the product as a colourless oil. (2.91 g, 95%). ¹H NMR (300 MHz, CDCl₃) 4.32 (2H, q), 3.08 (2H, m), 2.96 (1H, m), 2.14-1.22 (30H, m).

Step c. 4-(2-Adamantan-1-yl-ethyl)-2-cyclohexyl-thiazole-5-carboxylic acid. The product from step b (2.90 g, 7.35 mmol) was hydrolysed using essentially the procedure of Example 120, step c to afford the product as a white solid (1.80 g, 66%). ¹H NMR (300 MHz, d₆-DMSO) 13.00 (1H, br s), 2.95 (3H, m), 1.99-1.32 (27H, m).

Step d. 3-{[4-(2-Adamantan-1-yl-ethyl)-2-cyclohexyl-thiazole-5-carbonyl]-amino}-benzoic acid methyl ester. The product from step c was reacted with 3-amino-benzoic acid methyl ester using essentially the same method as in Example 52, step a. The product was obtained as a white solid in 59% yield. ¹H NMR (300 MHz, CDCl₃) 8.00 (2H, m), 7.82 (1H, d), 7.47 (2H, m), 3.93 (3H, s), 3.06 (3H, m), 2.18-1.27 (27H, m).

Step e. The product from step d (300 mg, 0.59 mmol) was hydrolysed using essentially the same method as in Example 118, step d, to afford the title compound as a white solid (246 mg, 90%). ¹H NMR (300 MHz, d₆-DMSO) 13.0 (1H, br s), 10.27 (1H, s), 8.28 (1H, s), 7.85 (1H, d), 7.66 (1H, d), 7.45 (1H, t), 2.90 (3H, m), 2.18-1.36 (27H, m). The acid was converted to the N-methyl-D-glucamine salt and lyophilised from water/dioxan. Found: C 60.27, H 8.01, N 5.93% C₃₆H₅₃N₃O₈S.1.5H₂O requires: C, 60.48; H, 7.90; N, 5.88%.

### EXAMPLE 136

(S)-3-{[5-(2-Adamantan-1-yl-ethyl)-2-pyrrolidin-2-yl-1H-imidazole-4-carbonyl]-amino}-benzoic Acid

Step a. (S)-5-(2-Adamantan-1-yl-ethyl)-2-[1-(9H-fluoren-9-ylmethoxycarbonyl)-pyrrolidin-2-yl]-]H-imidazole-4-carboxylic acid benzyl ester. 5-Adamantan-1-yl-2,3-dioxo-pentanoic acid benzyl ester monohydrate (Example 70, step a) (2.89 g, 7.77 mmol) was reacted with (S)-FMOC-pyrrolidine-2-carboxaldehyde (5.06 g, 15.54 mmol) using essentially the procedure of Example 20, step b. The crude product was purified by flash column chromatography (silica, DCM/ethyl acetate 4:1) to afford colourless oil (3.85 g, 76%). ¹H NMR (300 MHz, CDCl₃) (Signals are broad and complex due to restricted rotation.) 10.37 (1H, br d), 7.79-7.30 (13H, m), 5.30 (2H, m), 4.90 (1H, m), 4.45 (2H, m), 4.26 (1H, m), 3.50 (2H, m), 2.79 (2H, m, 2.20-1.35 (21H, m).

Step b. (S)-5-(2-Adamantan-1-yl-ethyl)-2-[1-(9H-fluoren-9-ylmethoxycarbonyl)-pyrrolidin-2-yl]-1H-imidazole-4-carboxylic acid. The product of step a (3.85 g, 5.88 mmol) was deprotected using the same procedure as in Example 1, step e to afford the acid as a white solid (3.05 g, 92%). ¹H NMR (300 MHz, d₆-DMSO) (Signals are broad and complex due to restricted rotation.) 12.40-11.90 (2H, br m), 7.87-7.06 (8H, m), 5.00-4.79 (1H, m), 4.25-4.03 (3H, m), 3.61 (1H, br s), 3.40 (1H, br s), 2.2-1.27 (21H, m).

Step c. (S)-3-{[5-(2-Adamantan-1-yl-ethyl)-2-[(1-(9H-fluoren-9-ylmethoxycarbonyl)-pyrrolidin-2-yl]-1H-imidazole-4-carbonyl]-amino}-benzoic acid benzyl ester. The product from step b (565 mg, 1 mmol) was reacted with 3-amino-benzoic acid benzyl ester (227 mg, 1 mmol) using essentially the procedure of Example 70, step c. The crude material was purified by flash column chromatography (silica, DCM/hexane/ethyl acetate, 2:2:1) to afford the product as a white solid (554 mg, 72%). ¹H NMR (300 MHz, CDCl₃) 10.33 (1H, s), 9.13 (1H, s), 8.24 (1H, d), 8.07 (1H, s), 7.78 (3H, d), 7.60 (2H, d), 7.50-7.30 (10H, m), 5.39 (2H, s), 4.94 (1H, m), 4.48 (2H, m), 4.28 (1H, t), 3.53 (2H, m), 2.96 (2H, m), 2.2-1.54 (19H, m), 1.38 (2H, m).

Step d. (S)-3-{[5-(2-Adamantan-]-yl-ethyl)-2-pyrrolidin-2-yl-1H-imidazole-4-carbonyl]-amino}-benzoic acid benzyl ester. To a solution of the product from step c (554 mg, 0.72 mmol) in DCM (4 ml) was added piperidine (1 ml). After standing for 30 min at room temperature the solution was diluted with DCM (20 ml) and washed with water (3×20 ml). The solution was dried (MgSO₄), and the solvent was evaporated. The residue was purified by flash column chromatography (silica, DCM/MeOH 9:1) to afford the product as a white solid (280 mg, 71%). ¹H NMR (300 MHz, CDCl₃) 10.00 (1H, br s), 9.09 (1H, s), 8.20 (1H, d), 8.11 (1H, s), 7.77 (1H, d), 7.45-7.34 (6H, m), 5.38 (2H, s), 4.39 (1H, t), 3.06 (4H, m), 2.24-1.70 (19H, m), 1.40 (2H, t).

Step e. The product from step d (100 mg, 0.18 mmol) was deprotected using essentially the same procedure as in Example 1, step e, with the modification that the hydrogenation was carried out for 2 h. The title compound was isolated as a white solid (72 mg, 87%). ¹H NMR (300 MHz, d₆-DMSO) 9.67 (1H, s), 8.40 (1H, s), 7.93 (1H, d), 7.62 (1H, d), 7.36 (1H, t), 4.40 (1H, t), 3.14 (2H, m), 2.90 (2H, m), 2.10-1.57 (19H, m), 1.38 (2H, m). The acid was converted to the N-methyl-D-glucamine salt and lyophilised from water/dioxan. Found: C, 58.77; H, 8.07; N, 10.03%. C₃₄H₅₁N₅O₈.2H₂O requires: C 58.85, H 7.99, N 10.09%.

### EXAMPLE 141

(±)-5-{[5-(2-Adamantan-1-yl-ethyl)-2-[1-methyl-piperidin-2-yl])-1H-imidazole-4-carbonyl]-amino}-2-methyl-benzoic Acid.

(±)-5-{[5-(2-Adamantan-1-yl-ethyl)-2-piperidin-2-yl-1H-imidazole-4-carbonyl]-amino}-2-methyl-benzoic acid benzyl was prepared using essentially the same procedure as in Example 136, steps a, b, c, d, with the modification that that (±)-FMOC-piperidine-2-carboxaldehyde was used instead of (S)-FMOC-pyrrolidine-2-carboxaldehyde in step a, and 5-amino-2-methyl-benzoic acid benzyl ester replaced 3-amino-benzoic acid benzyl ester in step c. The product was converted to the title compound following the procedure given in Example 137, steps a and b. ¹H NMR (300 MHz, d₆-DMSO) 12.20 (1H, br s), 9.53 (1H, s), 8.30 (1H, s), 7.76 (1H, d), 7.18 (1H, d), 3.2-2.88 (4H, m), 2.48 (3H, s), 2.45 (3H, s), 2.00-1.33 (24H, m). The acid was converted to the N-methyl-D-glucamine salt and lyophilised from water/dioxan. Found: C, 59.67; H, 8.51; N, 9.68%. C₃₇H₅₇N₅O₈.2H₂O requires: C 61.32, H 8.58, N 9.17%.

### EXAMPLE 216

3-{[5-(Adamantan-1-yloxymethyl)-2-cyclohexyl-H-imidazole-4-carbonyl]-amino}-benzoic Acid

Step a. (Adamantan-1-yloxy)-acetic acid. A mixture of (adamantan-1-yloxy)-acetic acid ethyl ester (A. F. Noels et al. Tetrahedron, 1982, 38, 2733) (7.29 g, 29 mmol) and potassium hydroxide (2.60 g, 46 mmol) in water-ethanol (1:2 mixture, 180 ml) was heated at reflux for 2 h. The mixture was cooled, then concentrated in vacuum and acidified with concentrated hydrochloric acid. The resultant white precipitate was dissolved in ethyl acetate (200 ml). The solution was washed with brine (2×200 ml), dried (MgSO₄) and the solvent was evaporated to afford white crystalline solid (5.85 g 92%). ¹H NMR (300 MHz, CDCl₃) 4.08 (2H, s), 2.20-1.58 (15H, m).

Step b. 4-(Adamantan-1-yloxy)-3-oxo-2-(triphenyl-1⁵-phosphanylidene)-butyric acid benzyl ester. Oxalyl chloride (18.6 ml, 0.214 mol) was added to a solution of the product of step a (39.13 g, 0.178 mol) in DCM (800 ml) containing catalytic amount of DMF at room temperature. The mixture was stirred at room temperature for 1 h, then the solvent was evaporated. The residue was dissolved in benzene (100 ml) and added dropwise to a solution of benzyl (triphenylphosphoranylidene)acetate (72.9 g, 0.178 mol) and N,O-bis(trimethylsilyl)acetamide (53.2 ml, 0.215 mol) in benzene (300 ml) at 0° C. The mixture was allowed to warm to room temperature, and stirred for 16 h. The reaction mixture was diluted with ethyl acetate (500 ml), washed with 5% aqueous potassium hydrogen sulfate (500 ml), 10% sodium carbonate (50 ml), brine (300 ml), dried (MgSO₄) and the solvent was evaporated. The residue was triturated with diethyl ether to afford white solid (93.83 g, 98%). ¹H NMR (300 MHz, CDCl₃) 7.64-6.94 (20H, m), 4.74 (2H, s), 4.72 (2H, s), 2.08-1.57 (15H, m).

Step c. 4-(Adamantan-1-yloxy)-2,3-dioxo-butyric acid benzyl ester monohydrate. To a vigorously stirred solution of the product of step b (12.0 g, 20.0 mmol) in DCM/water (1:1 mixture, 320 ml) were added tetrabutylammonium bromide (645 mg, 2.00 mmol) and potassium peroxymonosufate (OXONE) (24.7 g, 40.0 mmol) at 0° C. The mixture was stirred at room temperature for 48 h, the organic layer was separated, washed with water (3×100 ml), brine (100 ml), dried (MgSO₄) and the solvent was evaporated in vacuum. The residue was purified by flash chromatography (silica, hexane/ethyl acetate 1:1) to afford the product as pale yellow oil (6.1 g, 81%). ¹H NMR (300 MHz, CDCl₃) 7.33 (5H, m), 5.26 (2H, s), 4.98 (2H, br s), 4.30 (2H, s), 2.14 (3H, br s), 1.72-1.54 (12H, m).

Step d. 5-(Adamantan-1-yloxymethyl)-2-cyclohexyl-1H-imidazole-4-carboxylic acid benzyl ester. To a slurry of the product of step c (1.70 g, 4.54 mmol) and ammonium acetate (3.40 g, 45.4 mmol) in acetic acid (20 ml) was added cyclohexanecarboxaldehyde (1.10 ml 9.08 mmol). The mixture was stirred in an oil bath heated at 70° C. for 2 h. The solution was cooled to room temperature and the acetic acid was evaporated in vacuum. The residue was dissolved in ethyl acetate (30 ml), saturated sodium bicarbonate (100 ml) was slowly added and the mixture was stirred for 30 min. The organic layer was separated, washed with sodium bicarbonate (30 ml), brine (30 ml), dried (MgSO₄) and the solvent was evaporated. The crude product was purified by flash column chromatography (silica, DCM/ethyl acetate 4:1) to afford colourless foam (1.0 g, 49%). ¹H NMR (CDCl₃) 7.40 (5H, m), 5.30 (2H, s), 4.76 (2H, br s), 2.79 (1H, m), 2.14 (3H, br s), 2.05 (2H, m), 1.85-1.26 (20H, m).

Step e. 5-(Adamantan-1-yloxymethyl)-2-cyclohexyl-1H-imidazole-4-carboxylic acid. The product of step d (1.00 g, 2.23 mmol) was deprotected using the same procedure as in Example 1, step e to afford the acid as a white solid (0.76 g, 96%). ¹H NMR (300 MHz, d₆-DMSO) 12.00 (1H, br s), 4.60 (2H, br s), 2.63 (1H, m), 2.09 (3H, br s), 2.02-1.23 (22H, m).

Step f. 3-{[5-(Adamantan-1-yloxymethyl)-2-cyclohexyl-1H-imidazole-4-carbonyl]-amino}-benzoic acid benzyl ester. The product of step e (0.76 g, 2.12 mmol) was reacted with 3-amino-benzoic acid benzyl ester (0.48 g, 2.12 mmol) according to the procedure of Example 20, step d. The crude material was purified by flash chromatography (silica, DCM/ethyl acetate 98:2, then 95:5) to afford colourless foam (786 mg, 65.5%). ¹H NMR (300 MHz, CDCl₃) 9.50 and 8.90 (1H, 2×br s), 8.15 (2H, m), 7.81 (1H, d), 7.40 (6H, m), 5.38 (2H, s), 4.98 (2H, br s), 2.71 (1H, m), 2.16 (3H, br s), 2.00 (2H, m), 1.85-1.24 (20H, m).

Step g. The product of step f (780 mg, 1.37 mmol) was deprotected using the same procedure as in Example 1, step e to afford white solid (636 mg, 98%). ¹H NMR (300 MHz, d₆-DMSO) 12.50 (1H, br s), 12.00 (1H, br s), 9.70 (1H, br s), 8.45 (1H, s), 7.92 (1H, dd), 7.62 (1H, d), 7.41 (1H, t), 4.79 (2H, s), 2.70 (1H, m), 2.11 (3H, br s), 1.89 (2H, m), 1.76 (6H, m), 1.60 (10H, m), 1.30 (4H, m). The acid was converted to the N-methyl-D-glucamine salt and lyophilised from water/dioxan. Found: C, 58.06; H, 8.12; N, 7.62%. C₃₅H₅₂N₄O₉.3.0 mols H₂O requires: C, 57.84; H, 8.04; N, 7.71%. Alternatively, the acid was converted to the sodium salt. Found C, 64.83; H, 7.11; N, 8.06%. C₂₈H₃₄N₃O₄Na.1.07 mols H₂O requires C, 64.82; H, 7.02; N, 8.10%.

### EXAMPLE 217

5-{[5-(Adamantan-1-yloxymethyl)-2-cyclohexyl-1H-imidazole-4-carbonyl]-amino}-2-methyl-benzoic Acid

The title compound was prepared according to the procedure of Example 216, with the modification that 5-amino-2-methyl-benzoic acid benzyl ester was used in step f instead of 3-amino-benzoic acid benzyl ester. ¹H NMR (300 MHz, d₆— DMSO) 9.57 (1H, br s), 8.31 (1H, br s), 7.77 (1H, dd), 7.21 (1H, d), 4.78 (2H, s), 2.72 (1H, m), 2.46 (3H, s), 2.11 (3H, br s), 1.88 (2H, m), 1.79-1.54 (16H, m), 1.30 (4H, m). The acid was converted to the N-methyl-D-glucamine salt and lyophilised from water/dioxan. Found: C 56.95, H 8.38, N 7.41%. C₃₆H₅₄N₄O₉.4.0 mols H₂O requires: C, 56.98; H, 8.24; N, 7.38%.

### EXAMPLE 221

3-{[5-(Adamantan-1-yloxymethyl)-2-bicyclo[2.2.2]oct-1-yl-1H-imidazole-4-carbonyl]-amino}-benzoic Acid

The title compound was prepared according to the procedure of Example 216, with the modification that bicyclo[2.2.2]oct-1-yl-carbaldehyde (prepared by pyridinium chlorochromate oxidation of bicyclo[2.2.2]oct-1-yl-methanol (C. A. Grob, M. Ohta, E. Renk and A. Weiss, Helv. Chim. Acta 1958, 41, 1191)) was used in step d instead of cyclohexanecarboxaldehyde. ¹H NMR (300 MHz, d₆-DMSO) 12.80 (1H, br s), 12.06 (1H, br s), 9.59 (1H, s), 8.42 (111, s), 7.92 (1H, d), 7.60 (1H, d), 7.41 (1H, t), 4.77 (2H, s), 2.10 (3H, br s), 1.84 (6H, m), 1.76 (611, m), 1.64-1.53 (13H, m). The acid was converted to the N-methyl-D-glucamine salt and lyophilised from water/dioxan. Found: C 60.50, H 7.94, N 7.49%. C₃₇H₅₄N₄O₉.2.0 mols H₂O requires: C, 60.47; H, 7.96; N, 7.62%.

It is found that the compositions and products of the present invention comprising a compound of formula (I) and a proton pump inhibitor reduce hyperplasia, associated with administration of proton pump inhibitors. This was measured according to the following experimental protocol.

Animals and Treatment:

40 male SPF Wistar rats (200 g) were divided into 4 treatment groups and 2 strata. The treatment of the 20 rats in the second stratum started 2 weeks after the treatment of the first stratum. The design of the study was completely randomised double blind with individual blinding; all rats were placed in a separate cage. Animals had continuous access to water and food.

Animals were treated once daily during 14 days:

| | |
|---|---|
| Control | 1 ml s.c. 45% w/v β-hydroxypropylcyclodextrin (CD) + |
| group: | 1 ml p.o.(gavage) 0, 25% Methocel (Dow Corning) |
| PPI | 1 ml s.c. 45% w/v β-hydroxypropylcyclodextrin + |
| group: | 1 ml p.o.(gavage) 25 mg/kg Rabeprazole in 0.25% Methocel. |
| GRA | 1 ml s.c.160 mg/kg compound of ex. 216 in CD + |
| group: | 1 ml p.o. (gavage) 0, 25% Methocel. |
| GRA-PPI | 1 ml s.c.160 mg/kg compound of ex. 216 in CD + |
| group: | 1 ml p.o. (gavage) 25 mg/kg Rabeprazole in 0.25% Methocel. |

Preparation of Tissue:

After removal of the fundus, the stomach were rinsed with phosphate buffered saline prior to fixation with 4% formalin in Millonig buffer. After 4 hours immersion in fixative solutions at room temperature, tissue was rinsed in phosphate buffered saline (PBS), dehydrated and embedded in paraffin using the Leitz paraffin embedding station (Leitz TP 1050; Germany) dehydration module and paraffin embedding module (Leitz EG 1160; Germany).

Cross sections (3 μm thick) of the oxyntic part of the stomach were made at 3 levels, each separated by a distance of 400 μm.

Immunostaining

The following indirect immunofluorescence labeling method was used:

removal of paraffin and rehydratation of the sections followed by a blocking step

primary antibodies: polyclonal guinea pig anti-histidine decarboxylase, 1/2000 (from Euro-Diagnostica) and monoclonal mouse anti PCNA 1/2500 (Clone PC10 from Sigma). All antibodies were diluted in a 0.2% BSA solution. Sections were incubated overnight at 4° C. and then washed with a BSA solution.

secondary antibodies: goat anti guinea pig coupled to CY5, 1/500 (from Jackson Laboratories) and goat anti-mouse coupled to Cy3, 1/250 (from Jackson Laboratories); incubation for 4 hours at 37° C. After rinsing with BSA and PBS solutions, sections were mounted with slowfade (Molecular Probes Europe BV), and stored at 4° C.

Imaging

Fluorescence labelling was observed with an epifluorescence microscope or a Zeiss LSM510 (Carl Zeiss Jena GmbH) confocal microscope.

By using CY5- and CY3-coupled antibodies, the high autofluorescence properties of the oxyntic mucosa were circumvented when sections are illuminated by a 488 nm (FITC channel) light source. Negative controls, by omitting the primary antibodies, and an isotype control staining for PCNA showed complete absence of staining. The specific labelling of PCNA was checked using double staining with TOPRO-3® (Molecular Probes Europe BV), a nuclear stain. Only in the most luminal located epithelial cells, non-specific cytoplasmic labelling was present. In the glandular part of the mucosa, non-specific PCNA-staining was absent.

For determination of the labelling index of ECL cells, at least 80 confocal images per rat were taken from the 3 slides at the 3 different levels. The ratio of double labelled cells (HDC+PCNA) and all HDC labelled cells yielded the labelling index of ECL cells.

Proliferation activity of ECL cells in the PPI group is expected to be increased compared with sham, GRA and GRA-PPI groups (Eissele, R., Patberg, H., Koop, H., Krack, W., Lorenz, W., McKnight, A. T., and Arnold, R. Effect of gastrin receptor blockade on endrocine cells in rats during achlorhydria. Gastroenterology, 103, 1596-1601, 1992). Increased proliferation by PPI will be completely blocked by GRA.

[image]

FIG. 1 shows the labelling index of ECL cells after the two week treatment. Each point represents a single rat.

Using a Mann-Whitney test on the data it can be shown that the rebeprazole treated group is different to the control group with P=0.04. The same type of analysis shows that a significant difference exists when rebeprazole alone is compared to the group in which the compound of Example 216 is co-administered with rebeprazole, with a P value of 0.07 being obtained.

1. A pharmaceutical composition comprising a proton pump inhibitor and a compound of the formula (I) 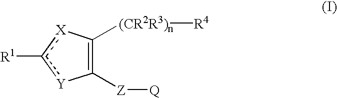
wherein X and Y are independently ═N—, —N(R⁵)— (R⁵ being selected from H, Me, Et, Pr, Bn, —OH and —CH₂COOR⁶, wherein R⁶ represents H, Me, Et, Pr or Bn), ═CH—, —S— or —O—
n is from 1 to 4;
R¹ is H or C₁ to C₁₅ hydrocarbyl wherein up to three C atoms may optionally be replaced by N, O and/or S atoms and up to three H atoms may optionally be replaced by halogen atoms;
R² is selected from H, Me, Et, Pr and OH, each R² being independently selected from H, Me, Et, Pr and OH when n is greater than 1;
R³ (when n is 1) is selected from H, Me, Et and Pr; or (when n is greater than 1) each R³ is independently selected from H, Me, Et and Pr, or two R³ groups on neighbouring carbon atoms are linked to form a C₃ to C₆ carbocylic ring, or two R³ groups are absent from neighbouring carbon atoms which are linked by a double bond; or R² and R³ on the same carbon atom together represent an ═O group;
R⁴ is C₁ to C₁₅ hydrocarbyl wherein up to two C atoms may optionally be replaced by N, O and/or S atoms and up to two H atoms may optionally be replaced by halogen atoms;
Z is —(NR⁷)ₐ—CO—(NR)_{b} (wherein a is 0 or 1, b is 0 or 1, and R⁷ and R⁸ are independently selected from the groups recited above for R⁶), —CO—NR⁷—CH₂—CO—NR⁵—, —CO—O—, —CH₂—CH₂—, —CH═CH₂, —CH═CH—, —CH═NR⁸— or a bond;
Q is —R⁹V, or 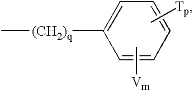
(wherein R⁹ is —CH₂—; —CH₂CH₂—; or 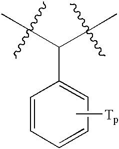
or R⁹ and R⁸, together with the nitrogen atom to which R⁸ is attached, form a piperidine or pyrrolidine ring which is substituted by V;
V is —CO—NH—SO₂-Ph, —SO₂—NH—CO-Ph, —CH₂OH, or a group of the formula —R¹⁰U, (wherein U is —COOH, tetrazolyl, —CONHOH— or —SO₃H; and R¹⁰ is a bond; C₁ to C₆ hydrocarbylene, optionally substituted by hydroxy, amino or acetamido; —O—(C₁ to C₃ alkylene)-; —SO₂NR¹¹—CHR¹²—;
—CO—NR¹¹—CHR¹²—, R¹¹ and R¹² being independently selected from H and methyl; or —NH—(CO)_{c}—CH₂—, c being 0 or 1);
T is C₁ to C₆ hydrocarbyl, —NR⁶R⁷ (wherein R⁶ and R⁷ are as defined above), —OMe, —OH, —CH₂OH, halogen or trihalomethyl;
m is 1 or 2;
p is from 0 to 3; and
q is from 0 to 2, with the proviso that q is 1 or 2 when Z is a bond);
or a pharmaceutically acceptable salt thereof,
together with a pharmaceutically acceptable diluent or carrier.

2. A composition according to claim 1 wherein in formula (I) Q is

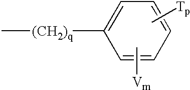

3. A composition according to claim 1 wherein in formula (I) Q is

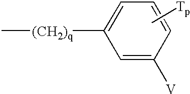

4. A composition according to any preceding claim wherein in formula (I) X and Y are independently ═N—, ═CH—, —NH—, —NOH— or —NMe-.

5. A composition according to claim 4 wherein in formula (I) X is —NH— and Y is ═CH—, or Y is
—NH— and X is —CH—.

6. A composition according to claim 4 wherein in formula (I) X is —NH— or —NOH— and Y is ═N— or wherein X is ═N— and Y is —NH— or —NOH—.

7. A composition according to any preceding claim wherein in formula (I) R¹ is C₁ to C₁₂ hydrocarbyl, wherein one C atom may optionally be replaced by N or O and up to three H atoms may optionally be replaced by F, Cl or Br.

8. A composition according to any preceding claim wherein in formula (I) R¹ is C₃ to C₁₂ alicyclic; phenyl (optionally substituted with OMe, NMe₂, CF₃, Me, F, Cl, Br or I); or C₁ to C₈ alkyl.

9. A composition according to any preceding claim wherein in formula (I) Z is —CO—NH—.

10. A composition according to any preceding claim wherein in formula (I) p is 0 or 1, and q is 0.

11. A compsition according to any preceding claim wherein in formula (I) T is C₁ to C₆ hydrocarbyl or halo.

12. A composition according to any preceding claim wherein in formula (I) V is —CO₂H, —CH₂CO₂H or tetrazolyl.

13. A composition according to any preceding claim wherein in formula (I) R² and R³ are H, and n is from 1 to 3.

14. A composition according to any of claims 1 to 12 wherein in formula (I) R² and R³ together form an ═O group, and n is 1.

15. A composition according to claim 13 or claim 14 wherein in formula (I) R⁴ is C₃ to C₁₂ carbocyclic, preferably adamantyl, cycloheptyl, cyclohexyl or phenyl.

16. A composition according to claim 13 or claim 14 wherein in formula (I) R⁴ is —NH—R¹³ or —OR¹³, in which R¹³ is C₃ to C₁₂ carbocyclic, preferably adamantyl, cycloheptyl, cyclohexyl or phenyl.

17. A composition according to claim 1 wherein in formula (I) R⁵ is selected from H, Me, Et, Pr and Bn; Z is —(NR⁷)ₐ—C—(NR⁸)_{b}—, —CO—NH—CH₂—CO—NH— or a bond; Q is 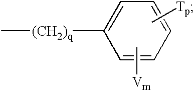
V is —CO—NH—SO₂-Ph, —SO₂—NH—CO-Ph, —OCH₂COOH, tetrazolyl or —(CH₂),COOH, wherein s is from 0 to 2; and T is C₁ to C₆ hydrocarbyl, —NR⁶R⁷, —OMe, —OH, —CH₂OH or halogen.

18. A composition according to claim 1 wherein in formula (I) R⁵ is selected from H, Me, Et, Pr and Bn; Z is —(NR⁷)ₐ—CO(NR⁸)_{b}—, Q is —CH₂)ᵣCOOH, wherein r is from 1 to 3; and T is C₁ to C₆ hydrocarbyl, —NR⁶R⁷, —OMe, —OH, —CH₂OH or halogen.

19. A composition according to claim 1 wherein in formula (I) R⁵ is selected from H, Me, Et, Pr and Bn; -Z-Q is 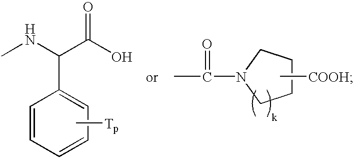
k is 1 or 2; and T is C₁ to C₆ hydrocarbyl, —NR⁶R⁷, —OMe, —OH, —CH₂OH or halogen.

20. A composition comprising a proton pump inhibitor and a compound which is degraded in vivo to yield a compound of formula (I) according to any preceding claim.

21. A composition according to any preceding claim wherein the proton pump inhibitor is selected from (RS)-rabeprazole, (RS)-omeprazole, lansoprazole, pantoprazole, (R)-omeprazole, (S)-omeprazole, perprazole, (R)-rabeprazole, (S)-rabeprazole, or the alkaline salts thereof.

22. A composition according to any preceding claim wherein the proton pump inhibitor and the compound of formula (I) are each in an amount producing a therapeutically beneficial effect in patients suffering from gastrointestinal disorders.

23. A composition according to claim 22 wherein said therapeutically beneficial effect is a synergistic effect on the reduction of acid secretion in patients suffering from gastrointestinal disorders, or the prevention of gastrointestinal disorders in said patients, or the reduction of adverse effects associated with the one of the active ingredients by the other active ingredients.

24. A composition according to any preceding claim wherein the amount of each of the active ingredients is equal to or less than that which is approved or indicated in monotherapy with said active ingredient.

25. A composition according to any preceding claim for use in medicine.

26. A composition according to any preceding claim for use in treating gastrointestinal disorders.

27. A product containing as first active ingredient a compound of formula (I) and as second active ingredient a proton pump inhibitor, as a combined preparation for simultaneous, separate or sequential use in the treatment of patients suffering from gastrointestinal disorders.

28. Use of a composition according to any one of claims 1 to 26 or a product according to claim 27 for the preparation of a medicament for the treatment of gastrointestinal disorders.

29. Use of a proton pump inhibitor for the preparation of a medicament for the treatment of gastrointestinal disorders, said treatment comprising the simultaneous or sequential administration of said proton pump inhibitor and a compound of formula (I), wherein said proton pump inhibitor enhances the effect of the compound of formula (I) on gastrin-related disorders in patients suffering from gastrointestinal disorders.

30. Use of a compound of formula (I) for the preparation of a medicament for the treatment of gastrointestinal disorders, said treatment comprising the simultaneous or sequential administration of said proton pump inhibitor and a compound of formula (I), wherein said compound of formula (I) enhances the effect of the proton pump inhibitor on the reduction of acid secretion in patients suffering from gastrointestinal disorders.

31. Use of a compound of formula (I) for the preparation of a medicament for reducing adverse effects associated with administration of proton pump inhibitors in patients suffering from gastrointestinal disorders.

32. Use according to claim 31 wherein the adverse effect is hyperplasia.

33. Use of a proton pump inhibitor for the preparation of a medicament for reducing adverse effects associated with administration of a compound of formula (I) in patients suffering from gastrointestinal disorders.

34. Use of a composition according to any one of claims 1 to 26 or a product according to claim 27 in the treatment of gastrointestinal disorders.

35. A method of making a pharmaceutical composition according to any one of claims 1 to 26, comprising mixing a compound of formula (I) and a proton pump inhibitor with a pharmaceutically acceptable diluent or carrier.
